(19) United States
(12) Patent Application Publication
(10) Pub. No.: US 2021/0107933 A1
(43) Pub. Date: Apr. 15, 2021

(54) HIGH-PURITY STEVIOL GLYCOSIDES

(71) Applicant: PureCircle USA Inc., Chicago, IL (US)

(72) Inventors: Avetik Markosyan, Yerevan (AM); Saravanan A/L Ramandach, Seremban (MY); Mohamad Afzaal Bin Hasim, Kuala Lumpur (MY); Khairul Nizam Bin Nawi, Nilai (MY); Siew Yin Chow, Klang (MY)

(73) Assignee: PureCircle USA Inc., Chicago, IL (US)

(21) Appl. No.: 16/981,677

(22) PCT Filed: Apr. 10, 2018
(86) PCT No.: PCT/US2018/026920

§ 371 Date: Sep. 16, 2020

Related U.S. Application Data
(60) Provisional application No. 62/644,065, filed on Mar. 16, 2018.
(60) Provisional application No. 62/644,407, filed on Mar. 17, 2018.

(51) Int. Cl. C07H 15/256; C12P 19/56; A61K 47/26; A61K 8/60; A23L 27/30; A23L 2/60

(52) U.S. Cl. CPC C07H 15/256; C12P 19/56; A61K 47/26; A23V 2002/00; A23L 27/36; A23L 2/60; A61K 8/602

(57) ABSTRACT

Methods of preparing highly purified steviol glycosides, particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, and rebaudioside AM are described. The methods include utilizing enzyme preparations and recombinant microorganisms for converting various staring compositions to target steviol glycosides. The highly purified rebaudiosides are useful as non-caloric sweetener, flavor enhancer, sweetness enhancer, and foaming suppressor in edible and chewable compositions such as any beverages, confectioneries, bakery products, cookies, and chewing gums.

### TECHNICAL FIELD

The present invention relates to a process for preparing compositions comprising steviol glycosides, including highly purified steviol glycoside compositions.

### BACKGROUND OF THE INVENTION

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling for their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10%-20% of the total dry weight. On a dry weight basis, the four major glycosides found in the leaves of Stevia typically include stevioside (9.1%), rebaudioside A (3.8%), rebaudioside C (0.6-1.0%) and dulcoside A (0.3%). Other known steviol glycosides include rebaudioside B, C, D, E, F and M, steviolbioside and rubusoside.

Although methods are known for preparing steviol glycosides from Stevia rebaudiana, many of these methods are unsuitable for use commercially.

Accordingly, there remains a need for simple, efficient, and economical methods for preparing compositions comprising steviol glycosides, including highly purified steviol glycoside compositions.

### SUMMARY OF THE INVENTION

The present invention provides a process for preparing a composition comprising a target steviol glycoside by contacting a starting composition comprising an organic substrate with a microbial cell and/or enzyme preparation, thereby producing a composition comprising a target steviol glycoside.

The starting composition can be any organic compound comprising at least one carbon atom. In one embodiment, the starting composition is selected from the group consisting of steviol glycosides, polyols or sugar alcohols, various carbohydrates.

The target steviol glycoside can be any steviol glycoside. In one embodiment, the target steviol glycoside is steviolmonoside, steviolmonoside A, steviolbioside, steviolbioside A, steviolbioside B, rubusoside, stevioside, stevioside A (rebaudioside K4), stevioside B, stevioside C, rebaudioside E, rebaudioside E2, rebaudioside E3, rebaudioside AM or a synthetic steviol glycoside.

In one embodiment, the target steviol glycoside is rebaudioside AM In some preferred embodiments enzyme preparation comprising one or more enzymes, or a microbial cell comprising one or more enzymes, capable of converting the starting composition to target steviol glycosides are used. The enzyme can be located on the surface and/or inside the cell. The enzyme preparation can be provided in the form of a whole cell suspension, a crude lysate or as purified enzyme(s). The enzyme preparation can be in free form or immobilized to a solid support made from inorganic or organic materials.

In some embodiments, a microbial cell comprises the necessary enzymes and genes encoding thereof for converting the starting composition to target steviol glycosides. Accordingly, the present invention also provides a process for preparing a composition comprising a target steviol glycoside by contacting a starting composition comprising an organic substrate with a microbial cell comprising at least one enzyme capable of converting the starting composition to target steviol glycosides, thereby producing a medium comprising at least one target steviol glycoside.

The enzymes necessary for converting the starting composition to target steviol glycosides include the steviol biosynthesis enzymes, UDP-glucosyltransferases (UGTs) and/or UDP-recycling enzyme.

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), l-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

As used hereinafter, the term “SuSy_AT”, unless specified otherwise, refers to sucrose synthase having amino-acid sequence “SEQ ID 1” as described in Example 1.

As used hereinafter, the term “UGTS12”, unless specified otherwise, refers to UDP-glucosyltransferase having amino-acid sequence “SEQ ID 2” as described in Example 1.

As used hereinafter, the term “UGT76G1”, unless specified otherwise, refers to UDP-glucosyltransferase having amino-acid sequence “SEQ ID 3” as described in Example 1.

In one embodiment, steviol biosynthesis enzymes and UDP-glucosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2, UGTS12, EUGT11 and UGTs having substantial (>85%, >86%, >87%, >88%, >89%, >90%, >91%, >92%, >93%, >94%, >95%, >96%,>97%, >98%, >99%) amino-acid sequence identity to these polypeptides as well as isolated nucleic acid molecules that code for these UGTs.

In one embodiment, steviol biosynthesis enzymes, UGTs and UDP-glucose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with UGT85C2.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with UGT74G1.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to the existing glucose at C13 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside to form steviolbioside.

In another embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside A (rebaudioside KA). In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside.

In another embodiment, the UDP-glucosyltransferase is any UDP-glucosyltransferase capable of adding at least one glucose unit to steviolbioside to form stevioside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside B to form rebaudioside E2.

In another embodiment, the UDP-glucosyltransferase is any UDP-glucosyltransferase capable of adding at least one glucose unit to stevioside A (rebaudioside KA) to form rebaudioside E3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A (rebaudioside KA) to form rebaudioside E.

In another embodiment, the UDP-glucosyltransferase is any UDP-glucosyltransferase capable of adding at least one glucose unit to stevioside C to form rebaudioside E3. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to stevioside to form rebaudioside E2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside to form rebaudioside E. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E3 to form rebaudioside AM.

In another embodiment, the UDP-glucosyltransferase is any UDP-glucosyltransferase capable of adding at least one glucose unit to rebaudioside E2 to form rebaudioside AM. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E to form rebaudioside AM In a particular embodiment, the UDP-glucosyltransferase is UGT76G1 or a UGT having >85% amino-acid sequence identity with UGT76G1.

Optionally, the method of the present invention further comprises using more than one UGT on a starting composition, to give a target steviol glycoside(s) having more than one glucose unit than the starting composition. In a particular embodiment, the UDP-glucosyltransferases are UGT74G1, UGT85C2, UGT76G1, UGTS12, EUGT11 and/or UGT91D2 or any UGT having >85% amino-acid sequence identity with UGT74G1, UGT85C2, UGT76G1, UGTS12, EUGT11 and/or UGT91D2 or any combination thereof, capable of adding more than one glucose unit to a starting composition to give a steviol glycoside(s) having more than one glucose unit than the starting composition.

In one embodiment, the UDP-glucosyltransferases are any UDP-glucosyltransferases capable of adding overall two glucose unit to stevioside to form rebaudioside AM. In a particular embodiment, the UDP-glucosyltransferases are selected from UGTS12, EUGT11, UGT91D2, UGT76G1 or any UGT having >85% amino-acid sequence identity with UGTS12, EUGT11, UGT91D2, UGT76G1 or any combination thereof. In another particular embodiment, the UDP-glucosyltransferases are UGTS12 and UGT76G1.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose.

In one embodiment, the recycling catalyst is sucrose synthase SuSy_At or a sucrose synthase having >85% amino-acid sequence identity with SuSy_At.

In one embodiment, the recycling substrate is sucrose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside AM.

Optionally, the method of the present invention further comprises separating the target steviol glycoside from the medium to provide a highly purified target steviol glycoside composition. The target steviol glycoside can be separated by at least one suitable method, such as, for example, crystallization, separation by membranes, centrifugation, extraction, chromatographic separation or a combination of such methods.

In one embodiment, the target steviol glycoside can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the conversion reaction.

In one embodiment, separation produces a composition comprising greater than about 80% by weight of the target steviol glycoside on an anhydrous basis, i.e., a highly purified steviol glycoside composition. In another embodiment, separation produces a composition comprising greater than about 90% by weight of the target steviol glycoside. In particular embodiments, the composition comprises greater than about 95% by weight of the target steviol glycoside. In other embodiments, the composition comprises greater than about 99% by weight of the target steviol glycoside.

The target steviol glycoside can be in any polymorphic or amorphous form, including hydrates, solvates, anhydrous or combinations thereof.

Purified target steviol glycosides can be used in consumable products as a sweetener, flavor modifier, flavor with modifying properties and/or foaming suppressor. Suitable consumable products include, but are not limited to, food, beverages, pharmaceutical compositions, tobacco products, nutraceutical compositions, oral hygiene compositions, and cosmetic compositions.

### BRIEF DESCRIPTION OF THE DRAWINGS

FIG. 1 shows the chemical structure of rebaudioside AM.

FIG. 2 shows the pathways of producing rebaudioside AM and various steviol glycosides from steviol.

FIG. 3 shows the biocatalytic production of rebaudioside AM from stevioside using the enzymes UGTS12 and UGT76G1 and concomitant recycling of UDP to UDP-glucose via sucrose synthase SuSy_At.

FIG. 4 shows the biocatalytic production of rebausioside AM from rebaudioside E using the enzyme UGT76G1 and concomitant recycling of UDP to UDP-glucose via sucrose synthase SuSy_At.

FIG. 5 shows the HPLC chromatogram of stevioside. The peak with retention time of 25.992 minutes corresponds to stevioside.

FIG. 6 shows the HPLC chromatogram of the product of the biocatalytic production of rebaudioside AM from stevioside. The peak with retention time of 10.636 minutes corresponds to rebaudioside AM.

FIG. 7 shows the HPLC chromatogram of rebaudioside E. The peak with retention time of 10.835 minutes corresponds to rebaudioside E.

FIG. 8 shows the HPLC chromatogram of the product of the biocatalytic production of rebaudioside AM from rebaudioside E. The peaks with retention time of 10.936 and 11.442 minutes correspond to rebaudioside E and rebaudioside AM respectively.

FIG. 9 shows the HPLC chromatogram of rebaudioside AM after purification by methanol crystallization. The peak with retention time of 10.336 minutes corresponds to rebaudioside AM.

FIG. 10 shows the ¹H NMR spectrum of rebaudioside AM (500 MHz, pyridine-d5).

FIG. 11 shows the HSQC spectrum of rebaudioside AM (500 MHz, pyridine-d5).

FIG. 12 shows the H,H COSY spectrum of rebaudioside AM (500 MHz, pyridine-d5).

FIG. 13 shows the HMBC spectrum of rebaudioside AM (500 MHz, pyridine-d5).

FIG. 14 shows the HSQC-TOCSY spectrum of rebaudioside AM (500 MHz, pyridine-d5).

FIG. 15a and FIG. 15b show the LC chromatogram and mass spectrum of rebaudioside AM respectively.

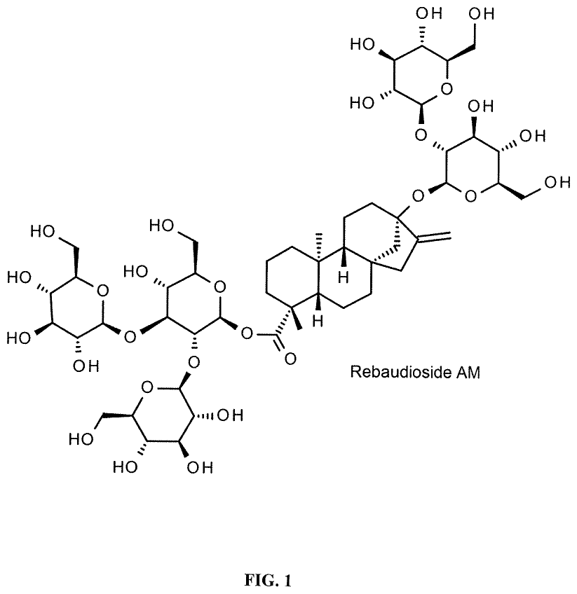

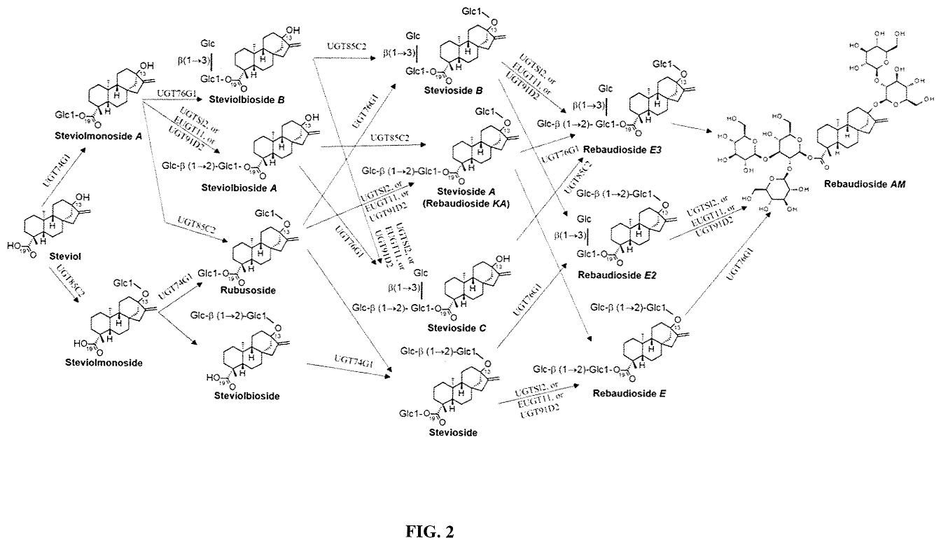

### DETAILED DESCRIPTION

The present invention provides a process for preparing a composition comprising a target steviol glycoside by contacting a starting composition comprising an organic substrate with a microbial cell and/or enzyme preparation, thereby producing a composition comprising a target steviol glycoside.

One object of the invention is to provide an efficient biocatalytic method for preparing target steviol glycosides, particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, rebaudioside AM or a synthetic steviol glycoside from various starting compositions.

As used herein, the abbreviation term “reb” refers to “rebaudioside”. Both terms have the same meaning and may be used interchangeably.

As used herein, “biocatalysis” or “biocatalytic” refers to the use of natural or genetically engineered biocatalysts, such as enzymes, or cells including microorganisms, comprising one or more enzyme, capable of single or multiple step chemical transformations on organic compounds. Biocatalysis processes include fermentation, biosynthesis, bioconversion and biotransformation processes. Both isolated enzyme, and whole-cell biocatalysis methods are known in the art. Biocatalyst protein enzymes can be naturally occurring or recombinant proteins.

As used herein, the term “steviol glycoside(s)” refers to a glycoside of steviol, including, but not limited to, naturally occurring steviol glycosides, e.g. steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, rebaudioside AM, synthetic steviol glycosides, e.g. enzymatically glucosylated steviol glycosides and combinations thereof.

Starting Composition

As used herein, “starting composition” refers to any composition (generally an aqueous solution) containing one or more organic compound comprising at least one carbon atom.

In one embodiment, the starting composition is selected from the group consisting of steviol, steviol glycosides, polyols and various carbohydrates.

The starting composition steviol glycoside is selected from the group consisting of steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 or other glycoside of steviol occurring in Stevia rebaudiana plant, synthetic steviol glycosides, e.g. enzymatically glucosylated steviol glycosides and combinations thereof.

In one embodiment, the starting composition is steviol.

In another embodiment, the starting composition steviol glycoside is steviolmonoside.

In yet another embodiment, the starting composition steviol glycoside is steviolmonoside A.

In still another embodiment, the starting composition steviol glycoside is rubusoside.

In yet another embodiment, the starting composition steviol glycoside is steviolbioside.

In yet another embodiment, the starting composition steviol glycoside is steviolbioside A.

In yet another embodiment, the starting composition steviol glycoside is steviolbioside B.

In still another embodiment, the starting composition steviol glycoside is stevioside.

In yet another embodiment, the starting composition steviol glycoside is stevioside A, also known as rebaudioside KA.

In still another embodiment, the starting composition steviol glycoside is stevioside B.

In still another embodiment, the starting composition steviol glycoside is stevioside C.

In another embodiment, the starting composition steviol glycoside is rebaudioside E.

In another embodiment, the starting composition steviol glycoside is rebaudioside E2.

In another embodiment, the starting composition steviol glycoside is rebaudioside E3.

The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced.

The term “carbohydrate” refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

The starting composition may be synthetic or purified (partially or entirely), commercially available or prepared.

In one embodiment, the starting composition is glycerol.

In another embodiment, the starting composition is glucose.

In still another embodiment, the starting composition is sucrose.

In yet another embodiment, the starting composition is starch.

In another embodiment, the starting composition is maltodextrin.

In yet another embodiment, the starting composition is cellulose.

In still another embodiment, the starting composition is amylose.

The organic compound(s) of starting composition serve as a substrate(s) for the production of the target steviol glycoside(s), as described herein.

Target Steviol Glycoside

The target steviol glycoside of the present method can be any steviol glycoside that can be prepared by the process disclosed herein. In one embodiment, the target steviol glycoside is selected from the group consisting of steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, rebaudioside AM or other glycoside of steviol occurring in Stevia rebaudiana plant, synthetic steviol glycosides, e.g. enzymatically glucosylated steviol glycosides and combinations thereof.

In one embodiment, the target steviol glycoside is steviolmonoside.

In another embodiment, the target steviol glycoside is steviolmonoside A.

In another embodiment, the target steviol glycoside is steviolbioside.

In another embodiment, the target steviol glycoside is steviolbioside A.

In another embodiment, the target steviol glycoside is steviolbioside B.

In another embodiment, the target steviol glycoside is rubusoside.

In another embodiment, the target steviol glycoside is stevioside.

In another embodiment, the target steviol glycoside is stevioside A (rebaudioside KA).

In another embodiment, the target steviol glycoside is stevioside B.

In another embodiment, the target steviol glycoside is stevioside C.

In another embodiment, the target steviol glycoside is rebaudioside E.

In another embodiment, the target steviol glycoside is rebaudioside E2.

In another embodiment, the target steviol glycoside is rebaudioside E3.

In another embodiment, the target steviol glycoside is rebaudioside AM The target steviol glycoside can be in any polymorphic or amorphous form, including hydrates, solvates, anhydrous or combinations thereof.

In one embodiment, the present invention is a biocatalytic process for the production of steviolmonoside.

In one embodiment, the present invention is a biocatalytic process for the production of steviolmonoside A.

In one embodiment, the present invention is a biocatalytic process for the production of steviolbioside.

In one embodiment, the present invention is a biocatalytic process for the production of steviolbioside A.

In one embodiment, the present invention is a biocatalytic process for the production of steviolbioside B.

In one embodiment, the present invention is a biocatalytic process for the production of rubusoside.

In one embodiment, the present invention is a biocatalytic process for the production of stevioside.

In one embodiment, the present invention is a biocatalytic process for the production of stevioside A (rebaudioside KA).

In one embodiment, the present invention is a biocatalytic process for the production of stevioside B.

In one embodiment, the present invention is a biocatalytic process for the production of stevioside C.

In one embodiment, the present invention is a biocatalytic process for the production of rebaudioside E.

In one embodiment, the present invention is a biocatalytic process for the production of rebaudioside E2.

In one embodiment, the present invention is a biocatalytic process for the production of rebaudioside E3.

In one embodiment, the present invention is a biocatalytic process for the production of rebaudioside AM.

In a particular embodiment, the present invention provides for the biocatalytic process for the production of rebaudioside AM from a starting composition comprising stevioside and UDP-glucose.

In another particular embodiment, the present invention provides for the biocatalytic process for the production of rebaudioside AM from a starting composition comprising rebaudioside E and UDP-glucose.

Optionally, the method of the present invention further comprises separating the target steviol glycoside from the medium to provide a highly purified target steviol glycoside composition. The target steviol glycoside can be separated by any suitable method, such as, for example, crystallization, separation by membranes, centrifugation, extraction, chromatographic separation or a combination of such methods.

In particular embodiments, the process described herein results in a highly purified target steviol glycoside composition. The term “highly purified”, as used herein, refers to a composition having greater than about 80% by weight of the target steviol glycoside on an anhydrous (dried) basis. In one embodiment, the highly purified target steviol glycoside composition contains greater than about 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is reb AM, the process described herein provides a composition having greater than about 90% reb AM content by weight on a dried basis. In another particular embodiment, when the target steviol glycoside is reb AM, the process described herein provides a composition comprising greater than about 95% reb AM content by weight on a dried basis.

Microorganisms and Enzyme Preparations

In one embodiment of present invention, a microorganism (microbial cell) and/or enzyme preparation is contacted with a medium containing the starting composition to produce target steviol glycosides.

The enzyme can be provided in the form of a whole cell suspension, a crude lysate, a purified enzyme or a combination thereof. In one embodiment, the biocatalyst is a purified enzyme capable of converting the starting composition to the target steviol glycoside. In another embodiment, the biocatalyst is a crude lysate comprising at least one enzyme capable of converting the starting composition to the target steviol glycoside. In still another embodiment, the biocatalyst is a whole cell suspension comprising at least one enzyme capable of converting the starting composition to the target steviol glycoside.

In another embodiment, the biocatalyst is one or more microbial cells comprising enzyme(s) capable of converting the starting composition to the target steviol glycoside. The enzyme can be located on the surface of the cell, inside the cell or located both on the surface of the cell and inside the cell.

Suitable enzymes for converting the starting composition to target steviol glycosides include, but are not limited to, the steviol biosynthesis enzymes and UDP-glucosyltransferases (UGTs). Optionally it may include UDP recycling enzyme(s).

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

In one embodiment, steviol biosynthesis enzymes and UDP-glucosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2, UGTS12, EUGT11 and UGTs having substantial (>85%, >86%, >87%, >88%, >89%, >90%, >91%, >92%, >93%, >94%, >95%, >96%,>97%, >98%, >99%) amino-acid sequence identity to these polypeptides as well as isolated nucleic acid molecules that code for these UGTs.

In one embodiment, steviol biosynthesis enzymes, UGTs and UDP-glucose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with UGT85C2.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with UGT74G1.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to the existing glucose at C13 of any starting steviol glycoside to give a target steviol glycoside with at least one additional glucose bearing at least one beta 1-42 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside to form steviolbioside.

In another embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside A (rebaudioside KA). In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside.

In another embodiment, the UDP-glucosyltransferase is any UDP-glucosyltransferase capable of adding at least one glucose unit to steviolbioside to form stevioside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside B to form rebaudioside E2.

In another embodiment, the UDP-glucosyltransferase is any UDP-glucosyltransferase capable of adding at least one glucose unit to stevioside A (rebaudioside KA) to form rebaudioside E3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A (rebaudioside KA) to form rebaudioside E.

In another embodiment, the UDP-glucosyltransferase is any UDP-glucosyltransferase capable of adding at least one glucose unit to stevioside C to form rebaudioside E3. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to stevioside to form rebaudioside E2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside to form rebaudioside E. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E3 to form rebaudioside AM.

In another embodiment, the UDP-glucosyltransferase is any UDP-glucosyltransferase capable of adding at least one glucose unit to rebaudioside E2 to form rebaudioside AM. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E to form rebaudioside AM. In a particular embodiment, the UDP-glucosyltransferase is UGT76G1 or a UGT having >85% amino-acid sequence identity with UGT76G1.

Optionally, the method of the present invention further comprises using more than one UGT on a starting composition, to give a target steviol glycoside(s) having more than one glucose unit than the starting composition. In a particular embodiment, the UDP-glucosyltransferases are UGT74G1, UGT85C2, UGT76G1, UGTS12, EUGT11 and/or UGT91D2 or any UGT having >85% amino-acid sequence identity with UGT74G1, UGT85C2, UGT76G1, UGTS12, EUGT11 and/or UGT91D2 or any combination thereof, capable of adding more than one glucose unit to a starting composition to give a steviol glycoside(s) having more than one glucose unit than the starting composition.

In one embodiment, the UDP-glucosyltransferases are any UDP-glucosyltransferases capable of adding overall two glucose unit to stevioside to form rebaudioside AM. In a particular embodiment, the UDP-glucosyltransferases are selected from UGTS12, EUGT11, UGT91D2, UGT76G1 or any UGT having >85% amino-acid sequence identity with UGTS12, EUGT11, UGT91D2, UGT76G1 or any combination thereof. In another particular embodiment, the UDP-glucosyltransferases are UGTS12 and UGT76G1.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose. The UDP recycling enzyme can be sucrose synthase SuSy_At or a sucrose synthase having >85% amino-acid sequence identity with SuSy_At and the recycling substrate can be sucrose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside AM.

In another embodiment, the UDP-glucosyltransferase capable of adding at least one glucose unit to starting composition steviol glycoside has >85% amino-acid sequence identity with UGTs selected from the following listing of GenInfo identifier numbers, preferably from the group presented in Table 1, and Table 2.

| | | | | | |
|---|---|---|---|---|---|
| 397567 | 30680413 | 115480946 | 147798902 | 218193594 | 225443294 |
| 454245 | 32816174 | 116310259 | 147811764 | 218193942 | 225444853 |
| 1359905 | 32816178 | 116310985 | 147827151 | 219885307 | 225449296 |
| 1685003 | 34393978 | 116788066 | 147836230 | 222615927 | 225449700 |
| 1685005 | 37993665 | 116788606 | 147839909 | 222619587 | 225454338 |
| 2191136 | 37993671 | 116789315 | 147846163 | 222623142 | 225454340 |
| 2501497 | 37993675 | 119394507 | 147855977 | 222625633 | 225454342 |
| 2911049 | 39104603 | 119640480 | 148905778 | 222625635 | 225454473 |
| 4218003 | 41469414 | 122209731 | 148905999 | 222636620 | 225454475 |
| 4314356 | 41469452 | 125526997 | 148906835 | 222636621 | 225458362 |
| 13492674 | 42566366 | 125534279 | 148907340 | 222636628 | 225461551 |
| 13492676 | 42570280 | 125534461 | 148908935 | 222636629 | 225461556 |
| 15217773 | 42572855 | 125540090 | 148909182 | 224053242 | 225461558 |
| 15217796 | 44890129 | 125541516 | 148909920 | 224053386 | 225469538 |
| 15223396 | 46806235 | 125545408 | 148910082 | 224055535 | 225469540 |
| 15223589 | 50284482 | 125547340 | 148910154 | 224056138 | 226316457 |
| 15227766 | 51090402 | 125547520 | 148910612 | 224056160 | 226492603 |
| 15230017 | 51090594 | 125554547 | 148910769 | 224067918 | 226494221 |
| 15231757 | 52839682 | 125557592 | 156138791 | 224072747 | 226495389 |
| 15234056 | 56550539 | 125557593 | 156138797 | 224080189 | 226495945 |
| 15234195 | 62734263 | 125557608 | 156138799 | 224091845 | 226502400 |
| 15234196 | 62857204 | 125559566 | 156138803 | 224094703 | 226507980 |
| 15238503 | 62857206 | 125563266 | 165972256 | 224100653 | 226531147 |
| 15239523 | 62857210 | 125571055 | 168016721 | 224100657 | 226532094 |
| 15239525 | 62857212 | 125579728 | 171674071 | 224101569 | 238477377 |
| 15239543 | 75265643 | 125588307 | 171906258 | 224103105 | 240254512 |
| 15239937 | 75285934 | 125589492 | 183013901 | 224103633 | 242032615 |
| 15240305 | 75288884 | 125599469 | 183013903 | 224103637 | 242032621 |
| 15240534 | 77550661 | 125601477 | 186478321 | 224109218 | 242038423 |
| 15982889 | 77556148 | 126635837 | 187373030 | 224114583 | 242043290 |
| 18086351 | 82791223 | 126635845 | 187373042 | 224116284 | 242044836 |
| 18418378 | 83778990 | 126635847 | 190692175 | 224120552 | 242051252 |
| 18418380 | 89953335 | 126635863 | 194701936 | 224121288 | 242056217 |
| 18418382 | 110741436 | 126635867 | 195620060 | 224121296 | 242056219 |
| 19743740 | 110743955 | 126635883 | 209954691 | 224121300 | 242056663 |
| 19911201 | 115438196 | 126635887 | 209954719 | 224130358 | 242059339 |
| 20149064 | 115438785 | 133874210 | 209954725 | 224140703 | 242059341 |
| 20260654 | 115441237 | 133874212 | 209954733 | 224143404 | 242060922 |
| 21435782 | 115454819 | 145358033 | 210063105 | 224143406 | 242067411 |
| 21553613 | 115456047 | 147772508 | 210063107 | 224144306 | 242067413 |
| 21593514 | 115457492 | 147776893 | 212275846 | 224285244 | 242076258 |
| 22759895 | 115459312 | 147776894 | 216296854 | 225431707 | 242076396 |
| 23955910 | 115464719 | 147776895 | 217074506 | 225435532 | 242084750 |
| 26452040 | 115471069 | 147786916 | 218185693 | 225436321 | 242091005 |
| 28393204 | 115471071 | 147798900 | 218187075 | 225440041 | 242095206 |
| 30679796 | 115474009 | 147798901 | 218189427 | 225441116 | 242345159 |
| 242345161 | 297724601 | 326492035 | 356523945 | 357140904 | 359486938 |
| 255536859 | 297725463 | 326493430 | 356523957 | 357165849 | 359487055 |
| 255538228 | 297728331 | 326500410 | 356523959 | 357165852 | 359488135 |
| 255541676 | 297738632 | 326506816 | 356523961 | 357168415 | 359488708 |
| 255547075 | 297745347 | 326507826 | 356523963 | 357437837 | 359493630 |
| 255552620 | 297745348 | 326508394 | 356524387 | 357442755 | 359493632 |
| 255552622 | 297795735 | 326509445 | 356524403 | 357442757 | 359493634 |
| 255555343 | 297796253 | 326511261 | 356527181 | 357445729 | 359493636 |
| 255555361 | 297796257 | 326511866 | 356533209 | 357445731 | 359493815 |
| 255555363 | 297796261 | 326512412 | 356533852 | 357445733 | 359495856 |
| 255555365 | 297797587 | 326517673 | 356534718 | 357446799 | 359495858 |
| 255555369 | 297798502 | 326518800 | 356535480 | 357446805 | 359495869 |
| 255555373 | 297799226 | 326521124 | 356542996 | 357452779 | 359495871 |
| 255555377 | 297805988 | 326525567 | 356543136 | 357452781 | 359497638 |
| 255556812 | 297807499 | 326525957 | 356543932 | 357452783 | 359807261 |
| 255556818 | 297809125 | 326526607 | 356549841 | 357452787 | 374256637 |
| 255563008 | 297809127 | 326527141 | 356549843 | 357452789 | 377655465 |
| 255564074 | 297811403 | 326530093 | 356554358 | 357452791 | 378405177 |
| 255564531 | 297820040 | 326534036 | 356554360 | 357452797 | 378829085 |
| 255572878 | 297821483 | 326534312 | 356558606 | 357452799 | 387135070 |
| 255577901 | 297825217 | 332071132 | 356560333 | 357470367 | 387135072 |
| 255583249 | 297832276 | 339715876 | 356560599 | 357472193 | 387135078 |
| 255583253 | 297832280 | 342306012 | 356560749 | 357472195 | 387135092 |
| 255583255 | 297832518 | 342306016 | 356566018 | 357474295 | 387135094 |
| 255585664 | 297832520 | 343457675 | 356566169 | 357474493 | 387135098 |
| 255585666 | 297840825 | 343457677 | 356566173 | 357474497 | 387135100 |
| 255634688 | 297840827 | 350534960 | 356567761 | 357474499 | 387135134 |
| 255644801 | 297847402 | 356498085 | 356574704 | 357490035 | 387135136 |
| 255645821 | 297849372 | 356499771 | 356576401 | 357493567 | 387135174 |
| 255647456 | 300078590 | 356499777 | 356577660 | 357497139 | 387135176 |
| 255648275 | 300669727 | 356499779 | 357114993 | 357497581 | 387135184 |
| 260279126 | 302142947 | 356501328 | 357115447 | 357497671 | 387135186 |
| 260279128 | 302142948 | 356502523 | 357115451 | 357500579 | 387135188 |
| 261343326 | 302142950 | 356503180 | 357115453 | 357504663 | 387135190 |
| 283132367 | 302142951 | 356503184 | 357116080 | 357504691 | 387135192 |
| 283362112 | 302765302 | 356503295 | 357116928 | 357504699 | 387135194 |
| 289188052 | 302796334 | 356504436 | 357117461 | 357504707 | 387135282 |
| 295841350 | 302811470 | 356504523 | 357117463 | 357505859 | 387135284 |
| 296088529 | 302821107 | 356504765 | 357117829 | 357510851 | 387135294 |
| 296090415 | 302821679 | 356511113 | 357117839 | 357516975 | 387135298 |
| 296090524 | 319759260 | 356515120 | 357125059 | 359477003 | 387135300 |
| 296090526 | 319759266 | 356517088 | 357126015 | 359477998 | 387135302 |
| 297599503 | 320148814 | 356520732 | 357134488 | 359478043 | 387135304 |
| 297601531 | 326489963 | 356522586 | 357135657 | 359478286 | 387135312 |
| 297611791 | 326490273 | 356522588 | 357138503 | 359484299 | 387135314 |
| 297722841 | 326491131 | 356522590 | 357139683 | 359486936 | 387135316 |
| 387135318 | 449440433 | 460376293 | 460413408 | 462423864 | 475546199 |
| 387135320 | 449445896 | 460378310 | 460416351 | 470101924 | 475556485 |
| 387135322 | 449446454 | 460380744 | 462394387 | 470102280 | 475559699 |
| 387135324 | 449447657 | 460381726 | 462394433 | 470102858 | 475578293 |
| 387135326 | 449449002 | 460382093 | 462394557 | 470104211 | 475591753 |
| 387135328 | 449449004 | 460382095 | 462395646 | 470104264 | 475593742 |
| 388493506 | 449449006 | 460382754 | 462395678 | 470104266 | 475612072 |
| 388495496 | 449451379 | 460384935 | 462396388 | 470106317 | 475622476 |
| 388498446 | 449451589 | 460384937 | 462396389 | 470106357 | 475622507 |
| 388499220 | 449451591 | 460385076 | 462396419 | 470115448 | 475623787 |
| 388502176 | 449451593 | 460385872 | 462396542 | 470130404 | 482550481 |
| 388517521 | 449453712 | 460386018 | 462397507 | 470131550 | 482550499 |
| 388519407 | 449453714 | 460389217 | 462399998 | 470136482 | 482550740 |
| 388521413 | 449453716 | 460394872 | 462400798 | 470136484 | 482550999 |
| 388827901 | 449453732 | 460396139 | 462401217 | 470136488 | 482552352 |
| 388827903 | 449457075 | 460397862 | 462402118 | 470136492 | 482554970 |
| 388827907 | 449467555 | 460397864 | 462402237 | 470137933 | 482555336 |
| 388827909 | 449468742 | 460398541 | 462402284 | 470137937 | 482555478 |
| 388827913 | 449495638 | 460403139 | 462402416 | 470140422 | 482556454 |
| 393887637 | 449495736 | 460403141 | 462404228 | 470140426 | 482557289 |
| 393887646 | 449499880 | 460403143 | 462406358 | 470140908 | 482558462 |
| 393887649 | 449502786 | 460403145 | 462408262 | 470141232 | 482558508 |
| 393990627 | 449503471 | 460405998 | 462409325 | 470142008 | 482558547 |
| 397746860 | 449503473 | 460407578 | 462409359 | 470142010 | 482561055 |
| 397789318 | 449515857 | 460407590 | 462409777 | 470142012 | 482561555 |
| 413924864 | 449518643 | 460409128 | 462411467 | 470143607 | 482562795 |
| 414590349 | 449519559 | 460409134 | 462414311 | 470143939 | 482562850 |
| 414590661 | 449522783 | 460409136 | 462414416 | 470145404 | 482565074 |
| 414591157 | 449524530 | 460409459 | 462414476 | 473923244 | 482566269 |
| 414879558 | 449524591 | 460409461 | 462415526 | 474114354 | 482566296 |
| 414879559 | 449528823 | 460409463 | 462415603 | 474143634 | 482566307 |
| 414879560 | 449528825 | 460409465 | 462415731 | 474202268 | 482568689 |
| 414888074 | 449534021 | 460409467 | 462416307 | 474299266 | 482570049 |
| 431812559 | 460365546 | 460410124 | 462416920 | 474363119 | 482570572 |
| 449432064 | 460366882 | 460410126 | 462416922 | 474366157 | 482575121 |
| 449432066 | 460369823 | 460410128 | 462416923 | 474429346 | |
| 449433069 | 460369829 | 460410130 | 462416924 | 475432777 | |
| 449436944 | 460369831 | 460410132 | 462417401 | 475473002 | |
| 449438665 | 460369833 | 460410134 | 462419769 | 475489790 | |
| 449438667 | 460370755 | 460410213 | 462420317 | 475511330 | |
| 449440431 | 460374714 | 460411200 | 462423366 | 475516200 | |

TABLE 1

| GI number | Accession | Origin |
|---|---|---|
| 190692175 | ACE87855.1 | Stevia rebaudiana |
| 41469452 | AAS07253.1 | Oryza saliva |
| 62857204 | BAD95881.1 | Ipomoea nil |
| 62857206 | BAD95882.1 | Ipomoea purperea |
| 56550539 | BAD77944.1 | Bellis perennis |
| 115454819 | NP_001051010.1 | Oryza sativa Japonica Group |
| 115459312 | NP_001053256.1 | Oryza sativa Japonica Group |
| 115471069 | NP_001059133.1 | Oryza saliva Japonica Group |
| 115471071 | NP_001059134.1 | Oryza saliva Japonica Group |
| 116310985 | CAH67920.1 | Oryza sativa Indica Group |
| 116788066 | ABK24743.1 | Picea sitchensis |
| 122209731 | Q2V6J9.1 | Fragaria × ananassa |
| 125534461 | EAY81009.1 | Oryza sativa Indica Group |
| 125559566 | EAZ05102.1 | Oryza sativa Indica Group |
| 125588307 | EAZ28971.1 | Oryza sativa Japonica Group |
| 148907340 | ABR16806.1 | Picea sitchensis |
| 148910082 | ABR18123.1 | Picea sitchensis |
| 148910612 | ABR18376.1 | Picea sitchensis |
| 15234195 | NP_194486.1 | Arabidopsis thaliana |
| 15239523 | NP_200210.1 | Arabidopsis thaliana |
| 15239937 | NP_196793.1 | Arabidopsis thaliana |
| 1685005 | AAB36653.1 | Nicotiana tabacum |
| 183013903 | ACC38471.1 | Medicago truncatula |
| 186478321 | NP_172511.3 | Arabidopsis thaliana |
| 187373030 | ACD03249.1 | Avena strigosa |
| 194701936 | ACF85052.1 | Zea mays |
| 19743740 | AAL92461.1 | Solanum lycopersicum |
| 212275846 | NP_001131009.1 | Zea mays |
| 222619587 | EEE55719.1 | Oryza sativa Japonica Group |
| 224055535 | XP_002298527.1 | Populus trichocarpa |
| 224101569 | XP_002334266.1 | Populus trichocarpa |
| 224120552 | XP_002318358.1 | Populus trichocarpa |
| 224121288 | XP_002330790.1 | Populus trichocarpa |
| 225444853 | XP_002281094 | Vitis vinifera |
| 225454342 | XP_002275850.1 | Vitis vinifera |
| 225454475 | XP_002280923.1 | Vitis vinifera |
| 225461556 | XP_002285222 | Vitis vinifera |
| 225469540 | XP_002270294.1 | Vitis vinifera |
| 226495389 | NP_001148083.1 | Zea mays |
| 226502400 | NP_001147674.1 | Zea mays |
| 238477377 | ACR43489.1 | Triticum aestivum |
| 240254512 | NP_565540.4 | Arabidopsis thaliana |
| 2501497 | Q43716.1 | Petunia × hybrida |
| 255555369 | XP_002518721.1 | Ricinus communis |
| 26452040 | BAC43110.1 | Arabidopsis thaliana |
| 296088529 | CBI37520.3 | Vitis vinifera |
| 297611791 | NP_001067852.2 | Oryza sativa Japonica Group |
| 297795735 | XP_002865752.1 | Arabidopsis lyrata subsp. lyrata |
| 297798502 | XP_002867135.1 | Arabidopsis lyrata subsp. lyrata |
| 297820040 | XP_002877903.1 | Arabidopsis lyrata subsp. lyrata |
| 297832276 | XP_002884020.1 | Arabidopsis lyrata subsp. lyrata |
| 302821107 | XP_002992218.1 | Selaginella moellendorffii |
| 30680413 | NP_179446.2 | Arabidopsis thaliana |
| 319759266 | ADV71369.1 | Pueraria montana var. lobata |
| 326507826 | BAJ86656.1 | Hordeum vulgare subsp. Vulgare |
| 343457675 | AEM37036.1 | Brassica rapa subsp. oleifera |
| 350534960 | NP_001234680.1 | Solanum lycopersicum |
| 356501328 | XP_003519477.1 | Glycine max |
| 356522586 | XP_003529927.1 | Glycine max |
| 356535480 | XP_003536273.1 | Glycine max |
| 357445733 | XP_003593144.1 | Medicago truncatula |
| 357452783 | XP_003596668.1 | Medicago truncatula |
| 357474493 | XP_003607531.1 | Medicago truncatula |
| 357500579 | XP_003620578.1 | Medicago truncatula |
| 357504691 | XP_003622634.1 | Medicago truncatula |
| 359477998 | XP_003632051.1 | Vitis vinifera |
| 359487055 | XP_002271587 | Vitis vinifera |
| 359495869 | XP_003635104.1 | Vitis vinifera |
| 387135134 | AFJ52948.1 | Linum usitatissimum |
| 387135176 | AFJ52969.1 | Linum usitatissimum |
| 387135192 | AFJ52977.1 | Linum usitatissimum |
| 387135282 | AFJ53022.1 | Linum usitatissimum |
| 387135302 | AFJ53032.1 | Linum usitatissimum |
| 387135312 | AFJ53037.1 | Linum usitatissimum |
| 388519407 | AFK47765.1 | Medicago truncatula |
| 393887646 | AFN26668.1 | Barbarea vulgaris subsp. arcuata |
| 414888074 | DAA64088.1 | Zea mays |
| 42572855 | NP_974524.1 | Arabidopsis thaliana |
| 449440433 | XP_004137989.1 | Cucumis sativus |
| 449446454 | XP_004140986.1 | Cucumis sativus |
| 449449004 | XP_004142255.1 | Cucumis sativus |
| 449451593 | XP_004143546.1 | Cucumis sativus |
| 449515857 | XP_004164964.1 | Cucumis sativus |
| 460382095 | XP_004236775.1 | Solanum lycopersicum |
| 460409128 | XP_004249992.1 | Solanum lycopersicum |
| 460409461 | XP_004250157.1 | Solanum lycopersicum |
| 460409465 | XP_004250159.1 | Solanum lycopersicum |
| 462396388 | EMJ02187.1 | Prunus persica |
| 462402118 | EMJ07675.1 | Prunus persica |
| 462409359 | EMJ14693.1 | Prunus persica |
| 462416923 | EMJ21660.1 | Prunus persica |
| 46806235 | BAD17459.1 | Oryza saliva Japonica Group |
| 470104266 | XP_004288529.1 | Fragaria vesca subsp. vesca |
| 470142008 | XP_004306714.1 | Fragaria vesca subsp. vesca |
| 475432777 | EMT01232.1 | Aegilops tauschii |
| 51090402 | BAD35324.1 | Oryza sativa Japonica Group |

TABLE 2

| | | | Internal |
|---|---|---|---|
| GI number | Accession | Origin | reference |
| 460409128 | XP.004249992.1 | Solanum lycopersicum | UGTSl |
| 460386018 | XP.004238697.1 | Solanum lycopersicum | — |
| 460409134 | XP.004249995.1 | Solanum lycopersicum | — |
| 460410132 | XP.004250485.1 | Solanum lycopersicum | UGTSl2 |
| 460410130 | XP.004250484.1 | Solanum lycopersicum | — |
| 460410128 | XP.004250483.1 | Solanum lycopersicum | — |
| 460378310 | XP.004234916.1 | Solanum lycopersicum | — |
| 209954733 | BAG80557.1 | Lycium barbarum | UGTLB |
| 209954725 | BAG80553.1 | Lycium barbarum | — |

One embodiment of the present invention is a microbial cell comprising an enzyme, i.e. an enzyme capable of converting the starting composition to the target steviol glycoside. Accordingly, some embodiments of the present method include contacting a microorganism with a medium containing the starting composition to provide a medium comprising at least one target steviol glycoside.

The microorganism can be any microorganism possessing the necessary enzyme(s) for converting the starting composition to target steviol glycoside(s). These enzymes are encoded within the microorganism's genome.

Suitable microoganisms include, but are not limited to, E. coli, Saccharomyces sp., Aspergillus sp., Pichia sp., Bacillus sp., Yarrowia sp. etc.

In one embodiment, the microorganism is free when contacted with the starting composition.

In another embodiment, the microorganism is immobilized when contacted with the starting composition. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

In still another embodiment, the enzyme capable of converting the starting composition to the target steviol glycoside is secreted out of the microorganism and into the reaction medium.

The target steviol glycoside is optionally purified. Purification of the target steviol glycoside from the reaction medium can be achieved by at least one suitable method to provide a highly purified target steviol glycoside composition. Suitable methods include crystallization, separation by membranes, centrifugation, extraction (liquid or solid phase), chromatographic separation, HPLC (preparative or analytical) or a combination of such methods.

Uses

Highly purified target glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention can be used “as-is” or in combination with other sweeteners, flavors, food ingredients and combinations thereof.

Non-limiting examples of flavors include, but are not limited to, lime, lemon, orange, fruit, banana, grape, pear, pineapple, mango, berry, bitter almond, cola, cinnamon, sugar, cotton candy, vanilla and combinations thereof.

Non-limiting examples of other food ingredients include, but are not limited to, acidulants, organic and amino acids, coloring agents, bulking agents, modified starches, gums, texturizers, preservatives, caffeine, antioxidants, emulsifiers, stabilizers, thickeners, gelling agents and combinations thereof.

Highly purified target glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention can be prepared in various polymorphic forms, including but not limited to hydrates, solvates, anhydrous, amorphous forms and combinations thereof.

Highly purified target glycoside(s) particularly, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

Highly purified target glycoside(s) particularly, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be employed as a sweetening compound as the sole sweetener, or it may be used together with at least one naturally occurring high intensity sweeteners such as rebaudioside A, rebaudioside A2, rebaudioside A3, rebaudioside B, rebaudioside C, rebaudioside C2, rebaudioside D, rebaudioside D2, rebaudioside F, rebaudioside F2, rebaudioside F3, rebaudioside G, rebaudioside H, rebaudioside I, rebaudioside I2, rebaudioside I3, rebaudioside J, rebaudioside K, rebaudioside K2, rebaudioside L, rebaudioside rebaudioside M2, rebaudioside N, rebaudioside O, rebaudioside O2, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside W, rebaudioside W2, rebaudioside W3, rebaudioside Y, rebaudioside Z1, rebaudioside Z2, dulcoside A, dulcoside C, stevioside D, stevioside E, stevioside E2, stevioside F, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside and combinations thereof.

In a particular embodiment, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be used in a sweetener composition comprising a compound selected from the group consisting of rebaudioside A, rebaudioside A2, rebaudioside A3, rebaudioside B, rebaudioside C, rebaudioside C2, rebaudioside D, rebaudioside D2, rebaudioside F, rebaudioside F2, rebaudioside F3, rebaudioside G, rebaudioside H, rebaudioside I, rebaudioside I2, rebaudioside I3, rebaudioside J, rebaudioside K, rebaudioside K2, rebaudioside L, rebaudioside M, rebaudioside M2, rebaudioside N, rebaudioside O, rebaudioside O2, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside W, rebaudioside W2, rebaudioside W3, rebaudioside Y, rebaudioside Z1, rebaudioside Z2, dulcoside A, dulcoside C, stevioside D, stevioside E, stevioside E2, stevioside F, NSF-02, Mogroside V, Luo Han Guo, allulose, allose, D-tagatose, erythritol and combinations thereof.

Highly purified target glycoside(s), particularly steviolmonoside, steviolmonoside A, steviolbioside, steviolbioside A, steviolbioside B, rubusoside, stevioside, stevioside A (rebaudioside K4), stevioside B, stevioside C, rebaudioside E, rebaudioside E2, rebaudioside E3 and/or rebaudioside AM may also be used in combination with synthetic high intensity sweeteners such as sucralose, potassium acesulfame, aspartame, alitame, saccharin, neohesperidin dihydrochalcone, cyclamate, neotame, dulcin, suosan advantame, salts thereof, and combinations thereof.

Moreover, highly purified target steviol glycoside(s) particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be used in combination with natural sweetener suppressors such as gymnemic acid, hodulcin, ziziphin, lactisole, and others. Steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may also be combined with various umami taste enhancers. Steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be mixed with umami tasting and sweet amino acids such as glutamate, aspartic acid, glycine, alanine, threonine, proline, serine, glutamate, lysine, tryptophan and combinations thereof.

Highly purified target steviol glycoside(s) particularly, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof.

Highly purified target steviol glycoside(s) particularly, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may be combined with reduced calorie sweeteners such as, for example, D-tagatose, L-sugars, L-sorbose, L-arabinose and combinations thereof.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may also be combined with various carbohydrates. The term “carbohydrate” generally refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, psicose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids; proteins; vitamins; and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be used as a sweetener not only for drinks, foodstuffs, and other products dedicated for human consumption, but also in animal feed and fodder with improved characteristics.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be applied as a flavor modifier to produce zero calorie, reduced calorie or diabetic beverages and food products with modified flavor. When used as a flavor modifier, or a flavor with modifying properties (FMP), the highly purified target steviol glycoside is used in a consumable product below the detection level of the flavor modifier or FMP. The flavor modifier or FMP therefore does not impart a detectable taste or flavor of its own to the consumable product, but instead serves to modify the consumer's detection of tastes and/or flavors of other ingredients in the consumable product. One example of taste and flavor modification is sweetness enhancement, in which the flavor modifier or FMP itself does not contribute to the sweetness of the consumable product, but enhances the quality of the sweetness tasted by the consumer.

Examples of consumable products in which highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may be used as a flavor modifier or flavor with modifying properties include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be applied as a foaming suppressor to produce zero calorie, reduced calorie or diabetic beverages and food products.

Examples of consumable products in which highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may be used as a sweetening compound include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

Moreover, the highly purified target steviol glycoside(s) steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained in this invention may be used in dry or liquid forms.

The highly purified target steviol glycoside can be added before or after heat treatment of food products. The amount of the highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM depends on the purpose of usage. As discussed above, it can be added alone or in combination with other compounds.

The present invention is also directed to sweetness enhancement in beverages using steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM. Accordingly, the present invention provides a beverage comprising a sweetener and steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM as a sweetness enhancer, wherein steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM is present in a concentration at or below their respective sweetness recognition thresholds.

As used herein, the term “sweetness enhancer” refers to a compound capable of enhancing or intensifying the perception of sweet taste in a composition, such as a beverage. The term “sweetness enhancer” is synonymous with the terms “sweet taste potentiator,” “sweetness potentiator,” “sweetness amplifier,” and “sweetness intensifier.”

The term “sweetness recognition threshold concentration,” as generally used herein, is the lowest known concentration of a sweet compound that is perceivable by the human sense of taste, typically around 1.0% sucrose equivalence (1.0% SE). Generally, the sweetness enhancers may enhance or potentiate the sweet taste of sweeteners without providing any noticeable sweet taste by themselves when present at or below the sweetness recognition threshold concentration of a given sweetness enhancer; however, the sweetness enhancers may themselves provide sweet taste at concentrations above their sweetness recognition threshold concentration. The sweetness recognition threshold concentration is specific for a particular enhancer and can vary based on the beverage matrix. The sweetness recognition threshold concentration can be easily determined by taste testing increasing concentrations of a given enhancer until greater than 1.0% sucrose equivalence in a given beverage matrix is detected. The concentration that provides about 1.0% sucrose equivalence is considered the sweetness recognition threshold.

In some embodiments, sweetener is present in the beverage in an amount from about 0.5% to about 12% by weight, such as, for example, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In a particular embodiment, the sweetener is present in the beverage in an amount from about 0.5% of about 10%, such as for example, from about 2% to about 8%, from about 3% to about 7% or from about 4% to about 6% by weight. In a particular embodiment, the sweetener is present in the beverage in an amount from about 0.5% to about 8% by weight. In another particular embodiment, the sweetener is present in the beverage in an amount from about 2% to about 8% by weight.

In one embodiment, the sweetener is a traditional caloric sweetener. Suitable sweeteners include, but are not limited to, sucrose, fructose, glucose, high fructose corn syrup and high fructose starch syrup.

In another embodiment, the sweetener is erythritol.

In still another embodiment, the sweetener is a rare sugar. Suitable rare sugars include, but are not limited to, D-allose, D-psicose, D-ribose, D-tagatose, L-glucose, L-fucose, L-arabinose, D-turanose, D-leucrose and combinations thereof.

It is contemplated that a sweetener can be used alone, or in combination with other sweeteners.

In one embodiment, the rare sugar is D-allose. In a more particular embodiment, D-allose is present in the beverage in an amount of about 0.5% to about 10% by weight, such as, for example, from about 2% to about 8%.

In another embodiment, the rare sugar is D-psicose. In a more particular embodiment, D-psicose is present in the beverage in an amount of about 0.5% to about 10% by weight, such as, for example, from about 2% to about 8%.

In still another embodiment, the rare sugar is D-ribose. In a more particular embodiment, D-ribose is present in the beverage in an amount of about 0.5% to about 10% by weight, such as, for example, from about 2% to about 8%.

In yet another embodiment, the rare sugar is D-tagatose. In a more particular embodiment, D-tagatose is present in the beverage in an amount of about 0.5% to about 10% by weight, such as, for example, from about 2% to about 8%.

In a further embodiment, the rare sugar is L-glucose. In a more particular embodiment, L-glucose is present in the beverage in an amount of about 0.5% to about 10% by weight, such as, for example, from about 2% to about 8%.

In one embodiment, the rare sugar is L-fucose. In a more particular embodiment, L-fucose is present in the beverage in an amount of about 0.5% to about 10% by weight, such as, for example, from about 2% to about 8%.

In another embodiment, the rare sugar is L-arabinose. In a more particular embodiment, L-arabinose is present in the beverage in an amount of about 0.5% to about 10% by weight, such as, for example, from about 2% to about 8%.

In yet another embodiment, the rare sugar is D-turanose. In a more particular embodiment, D-turanose is present in the beverage in an amount of about 0.5% to about 10% by weight, such as, for example, from about 2% to about 8%.

In yet another embodiment, the rare sugar is D-leucrose. In a more particular embodiment, D-leucrose is present in the beverage in an amount of about 0.5% to about 10% by weight, such as, for example, from about 2% to about 8%.

The addition of the sweetness enhancer at a concentration at or below its sweetness recognition threshold increases the detected sucrose equivalence of the beverage comprising the sweetener and the sweetness enhancer compared to a corresponding beverage in the absence of the sweetness enhancer. Moreover, sweetness can be increased by an amount more than the detectable sweetness of a solution containing the same concentration of the at least one sweetness enhancer in the absence of any sweetener.

Accordingly, the present invention also provides a method for enhancing the sweetness of a beverage comprising a sweetener comprising providing a beverage comprising a sweetener and adding a sweetness enhancer selected from steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM or a combination thereof, wherein steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM are present in a concentration at or below their sweetness recognition thresholds.

Addition of steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM in a concentration at or below the sweetness recognition threshold to a beverage containing a sweetener may increase the detected sucrose equivalence from about 1.0% to about 5.0%, such as, for example, about 1.0%, about 1.5%, about 2.0%, about 2.5%, about 3.0%, about 3.5%, about 4.0%, about 4.5% or about 5.0%.

The following examples illustrate preferred embodiments of the invention for the preparation of highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM. It will be understood that the invention is not limited to the materials, proportions, conditions and procedures set forth in the examples, which are only illustrative.

### EXAMPLES

### Example 1

Protein Sequences of Engineered Enzymes Used in the Biocatalytic Process

SEQ ID 1:
>SuSy_At, variant PM1-54-2-E05 (engineered
sucrose synthase; source of WT gene:
Arabidopsis thaliana)
MANAERMITRVHSQRERLNETLVSERNEVLALLSRVEAKGKGILQQNQII
AEFEALPEQTRKKLEGGPFFDLLKSTQEAIVLPPWVALAVRPRPGVWEYL
RVNLHALVVEELQPAEFLHFKEELVDGVKNGNFTLELDFEPFNASIPRPT
LHKYIGNGVDFLNRHLSAKLFHDKESLLPLLDFLRLHSHQGKNLMLSEKI
QNLNTLQHTLRKAEEYLAELKSETLYEEFEAKFEEIGLERGWGDNAERVL
DMIRLLLDLLEAPDPSTLETFLGRVPMVFNVVILSPHGYFAQDNVLGYPD
TGGQVVYILDQVRALEIEMLQRIKQQGLNIKPRILILTRLLPDAVGTTCG
ERLERVYDSEYCDILRVPFRTEKGIVRKWISRFEVWPYLETYTEDAAVEL
SKELNGKPDLIIGNYSDGNLVASLLAHKLGVTQCTIAHALEKTKYPDSDI
YWKKLDDKYHFSCQFTADIFAMNHTDFIITSTFQEIAGSKETVGQYESHT
AFTLPGLYRVVHGIDVFDPKFNIVSPGADMSIYFPYTEEKRRLTKFHSEI
EELLYSDVENDEHLCVLKDKKKPILFTMARLDRVKNLSGLVEWYGKNTRL
RELVNLVVVGGDRRKESKDNEEKAEMKKMYDLIEEYKLNGQFRWISSQMD
RVRNGELYRYICDTKGAFVQPALYEAFGLTVVEAMTCGLPTFATCKGGPA
EIIVHGKSGFHIDPYHGDQAADLLADFFTKCKEDPSHWDEISKGGLQRIE
EKYTWQIYSQRLLTLTGVYGFWKHVSNLDRLEHRRYLEMFYALKYRPLAQ
AVPLAQDD
SEQ ID 2:
>UGTS12 variant 0234 (engineered
glucosyltransferase; source of WT gene:
Solanum lycopersicum)
MATNLRVLMFPWLAYGHISPFLNIAKQLADRGFLIYLCSTRINLESIIKK
IPEKYADSIHLIELQLPELPELPPHYHTTNGLPPHLNPTLHKALKMSKPN
FSRILQNLKPDLLIYDVLQPWAEHVANEQGIPAGKLLVSCAAVFSYFFSF
RKNPGVEFPFPAIHLPEVEKVKIREILAKEPEEGGRLDEGNKQMMLMCTS
RTIEAKYIDYCTELCNWKVVPVGPPFQDLITNDADNKELIDWLGTKPENS
TVFVSFGSEYFLSKEDMEEIAFALEASNVNFIWVVRFPKGEERNLEDALP
EGFLERIGERGRVLDKFAPQPRILNHPSTGGFISHCGWNSVMESIDFGVP
IIAMPIHNDQPINAKLMVELGVAVEIVRDDDGKIHRGEIAEALKSVVTGE
TGEILRAKVREISKNLKSIRDEEMDAVAEELIQLCRNSNKSK
SEQ ID 3:
>UGT76G1 variant 0042 (engineered
glucosyltransferase; source of WT gene:
Stevia rebaudiana)
MENKTETTVRRRRRIILFPVPFQGHINPILQLANVLYSKGFAITILHTNF
NKPKTSNYPHFTFRFILDNDPQDERISNLPTHGPLAGMRIPIINEHGADE
LRRELELLMLASEEDEEVSCLITDALWYFAQDVADSLNLRRLVLMTSSLF
NFHAHVSLPQFDELGYLDPDDKTRLEEQASGFPMLKVKDIKSAYSNWQIG
KEILGKMIKQTKASSGVIWNSFKELEESELETVIREIPAPSFLIPLPKHL
TASSSSLLDHDRTVFEWLDQQAPSSVLYVSFGSTSEVDEKDFLEIARGLV
DSGQSFLWVVRPGFVKGSTWVEPLPDGFLGERGKIVKWVPQQEVLAHPAI
GAFWTHSGWNSTLESVCEGVPMIFSSFGGDQPLNARYMSDVLRVGVYLEN
GWERGEVVNAIRRVMVDEEGEYIRQNARVLKQKADVSLMKGGSSYESLES
LVSYISSL

### Example 2

Expression and Formulation of SuSy_At Variant of SEQ ID 1

The gene coding for the SuSy_At variant of SEQ ID 1 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.2 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with distilled water, resulting in an enzymatic active preparation.

For enzymatic active preparations of SuSy_At, activity in Units is defined as follows: 1 mU of SuSy_At turns over 1 nmol of sucrose into fructose in 1 minute. Reaction conditions for the assay are 30° C., 50 mM potassium phosphate buffer pH 7.0, 400 mM sucrose at t₀, 3 mM MgCl₂, and 15 mM uridine diphosphate (UDP).

### Example 3

Expression and Formulation of UGTS12 Variant of SEQ ID 2

The gene coding for the UGTS12 variant of SEQ 1D 2 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/1) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGTS12, activity in Units is defined as follows: 1 mU of UGTS12 turns over 1 nmol of rebaudioside A (RebA) into rebaudioside D (Reb D) in 1 minute. Reaction conditions for the assay are 30° C., 50 mM potassium phosphate buffer pH 7.0, 10 mM RebA at t₀, 500 mM sucrose, 3 mM MgCl₂, 0.25 mM uridine diphosphate (UDP) and 3 U/mL of SuSy_At.

### Example 4

Expression and Formulation of UGT76G1 Variant of SEQ ID 3

The gene coding for the UGT76G1 variant of SEQ ID 3 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/I) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGT76G1, activity in Units is defined as follows: 1 mU of UGT76G1 turns over 1 nmol of rebaudioside D (Reb D) into rebaudioside M (Reb M) in 1 minute. Reaction conditions for the assay are 30° C., 50 mM potassium phosphate buffer pH 7.0, 10 mM RebA at t₀, 500 mM sucrose, 3 mM MgCl₂, 0.25 mM uridine diphosphate (UDP) and 3 U/mL of SuSy_At.

### Example 5

Synthesis of Rebaudioside AM from Stevioside in a One-Pot Reaction, Adding UGTS12, SuSy_At and UGT76G1 at the Same Time

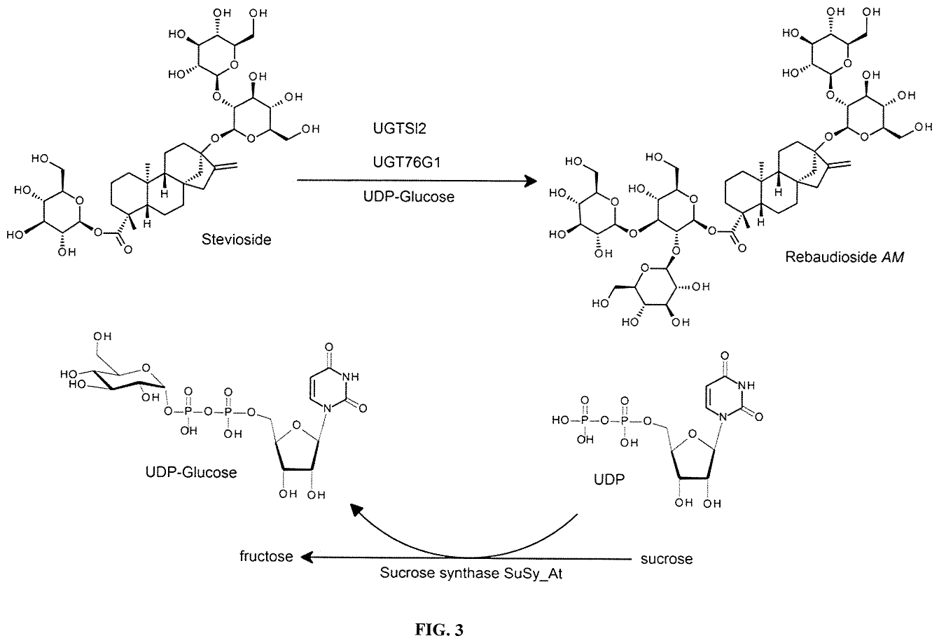

Rebaudioside AM (reb AM) was synthesized directly from stevioside in a one-pot reaction (FIG. 3), utilizing the three enzymes (see EXAMPLES 1, 2, 3 and 4): UGTS12 (variant of SEQ ID 2), SuSy_At-(variant of SEQ ID 1) and UGT76G1 (variant of SEQ ID 3). The final reaction solution contained 105 U/L UGTS12, 405 U/L SuSy_At, 3 U/L UGT76G1, 5 mM stevioside, 0.25 mM uridine diphosphate (UDP), 1 M sucrose, 4 mM MgCl₂ and potassium phosphate buffer (pH 6.6). First, 207 mL of distilled water were mixed with 0.24 g MgCl₂.6H₂O, 103 g sucrose, 9.9 mL of 1.5 M potassium phosphate buffer (pH 6.6) and 15 g stevioside. After dissolving the components, the temperature was adjusted to 45° C. and UGTS12, SuSy_At, UGT76G1 and 39 mg UDP were added. The reaction mixture was incubated at 45° C. shaker for 24 hrs. Additional 39 mg UDP was added at 8 hrs and 18 hours. The content of reb AM, reb E, stevioside, reb M, reb B, steviolbioside and reb/at several time points was analyzed by HPLC.

For analysis, biotransformation samples were inactivated by adjusting the reaction mixture to pH5.5 using 17% H₃PO₄ and then boiled for 10 minutes. Resulting samples were filtered, the filtrates were diluted 10 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB— C18, 4.6 mm×150 mm, 2.7 μm at 40° C. The mobile phase consisted of two premixes:
premix 1 containing 75% 10 mM phosphate buffer (pH2.6) and 25% acetonitrile, and
premix 2 containing 68% 10 mM phosphate buffer (pH2.6) and 32% acetonitrile.

Elution gradient started with premix 1, changed to premix 2 to 50% at 12.5 minute, changed to premix 2 to 100% at 13 minutes. Total run time was 45 minutes. The column temperature was maintained at 40° C. The injection volume was 5 μL. Rebaudioside species were detected by UV at 210 nm.

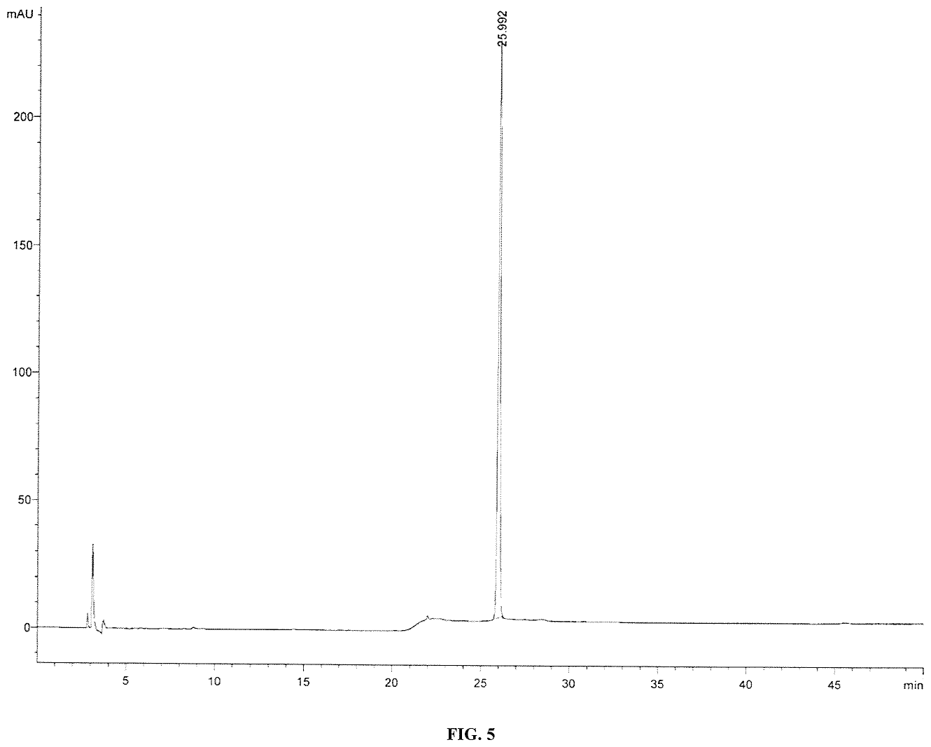

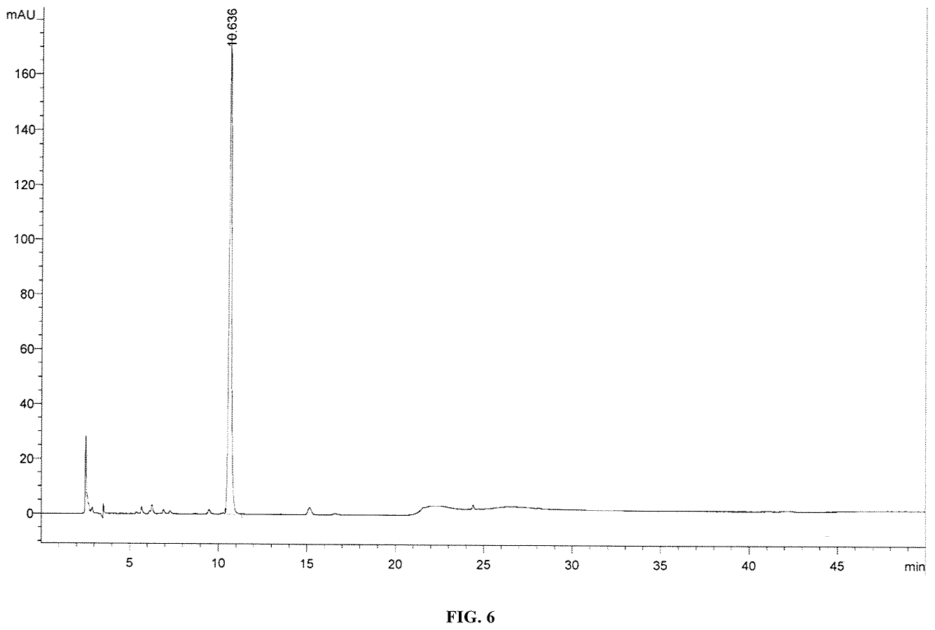

Table 3 shows for each time point the conversion of stevioside into identified rebaudioside species (area percentage). The chromatograms of stevioside and the reaction mixture at 24 hours are shown in FIG. 5 and FIG. 6, respectively. Those with skill in the art will appreciate that retention times can occasionally vary with changes in solvent and/or equipment.

TABLE 3
Biotransformation of stevioside to reb AM

| Time, | % conversion from stevioside | | | | | | |
|---|---|---|---|---|---|---|---|
| hrs | Reb E | Reb AM | Reb M | Reb I | Stevioside | Reb B | Steviolbioside |
| 0 | 0 | 0 | 0 | 0 | 100 | 0 | 0 |
| 6 | 1.9 | 35.9 | 1.3 | 1.7 | 58.7 | 0.0 | 0.4 |
| 18 | 0.9 | 96.7 | 1.3 | 0.6 | 0.0 | 0.0 | 0.4 |
| 24 | 0.3 | 96.4 | 2.1 | 0.7 | 0.0 | 0.2 | 0.4 |

### Example 6

Synthesis of Rebaudioside AM from Rebaudioside E in a One-Pot Reaction, SuSy_At and UGT76G1 at the Same Time

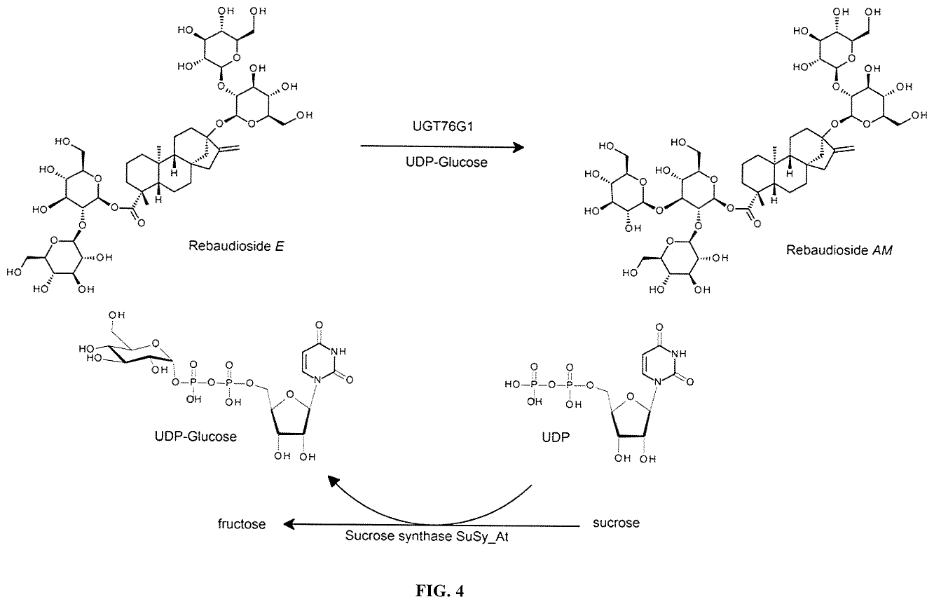

Rebaudioside AM (reb AM) was synthesized directly from rebaudioside E (reb E) in a one-pot reaction (FIG. 4), utilizing the two enzymes (see EXAMPLES 1, 2 and 4): SuSy_At-(variant of SEQ ID 1) and UGT76G1 (variant of SEQ ID 3). The final reaction solution contained 405 U/L SuSy_At, 3 U/L UGT76G1, 5 mM reb E, 0.25 mM uridine diphosphate (UDP), 1 M sucrose, 4 mM MgCl₂.6H₂O and potassium phosphate buffer (pH 6.6). First, 37 mL of distilled water were mixed with 40.3 mg MgCl₂, 17.12 g sucrose, 1.65 mL of 1.5 M potassium phosphate buffer (pH 6.6) and 5.04 g reb E. After dissolving the components, the temperature was adjusted to 45° C. and SuSy_At, UGT76G1 and 6.5 mg UDP were added. The reaction mixture was incubated at 45° C. shaker for 24 hrs. Additional 6.5 mg UDP was added at 8 hrs and 18 hours. The content of reb AM, reb E, stevioside, reb A, reb M, reb B, and steviolbioside at several time points was analyzed by HPLC.

For analysis, biotransformation samples were inactivated by adjusting the reaction mixture to pH5.5 using 17% H₃PO₄ and then boiled for 10 minutes. Resulting samples were filtered, the filtrates were diluted 10 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB— C18, 4.6 mm×150 mm, 2.7 μm at 40° C. The mobile phase consisted of two premixes:
premix 1 containing 75% 10 mM phosphate buffer (pH2.6) and 25% acetonitrile, and
premix 2 containing 68% 10 mM phosphate buffer (pH2.6) and 32% acetonitrile.

Elution gradient started with premix 1, changed to premix 2 to 50% at 12.5 minute, changed to premix 2 to 100% at 13 minutes. Total run time was 45 minutes. The column temperature was maintained at 40° C. The injection volume was 5 μL. Rebaudioside species were detected by UV at 210 nm.

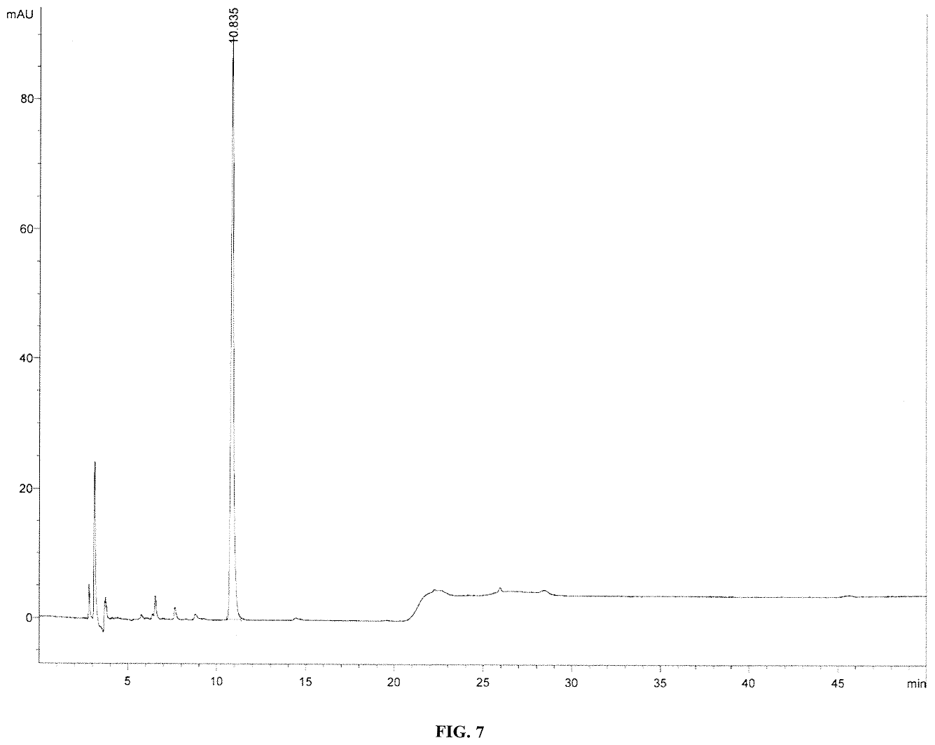

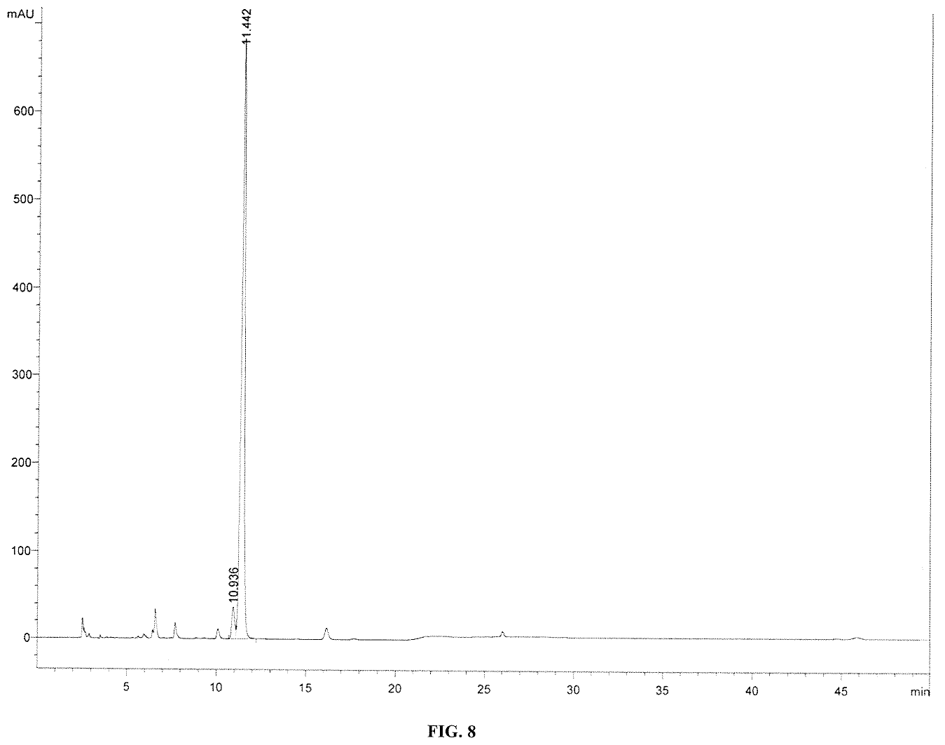

Table 4 shows for each time point the conversion of reb E into identified rebaudioside species (area percentage). The chromatograms of reb E and the reaction mixture at 24 hours are shown in FIG. 7 and FIG. 8, respectively. Those with skill in the art will appreciate that retention times can occasionally vary with changes in solvent and/or equipment.

TABLE 4
Biotransformation of reb E to reb AM

| | % conversion from Reb E | | | | | | |
|---|---|---|---|---|---|---|---|
| Time, | Reb | Reb | Reb | Reb | | Reb | Steviol- |
| hrs | E | AM | M | A | Stevioside | B | bioside |
| 0 | 99.46 | 0 | 0 | 0.54 | 0 | 0 | 0 |
| 4 | 40.75 | 57.92 | 0 | 0.59 | 0 | 0.73 | 0 |
| 7 | 24.79 | 73.92 | 0 | 0.58 | 0 | 0.71 | 0 |
| 24 | 4.38 | 94.33 | 0 | 0.59 | 0 | 0.70 | 0 |

### Example 7

Purification of Rebaudioside AM

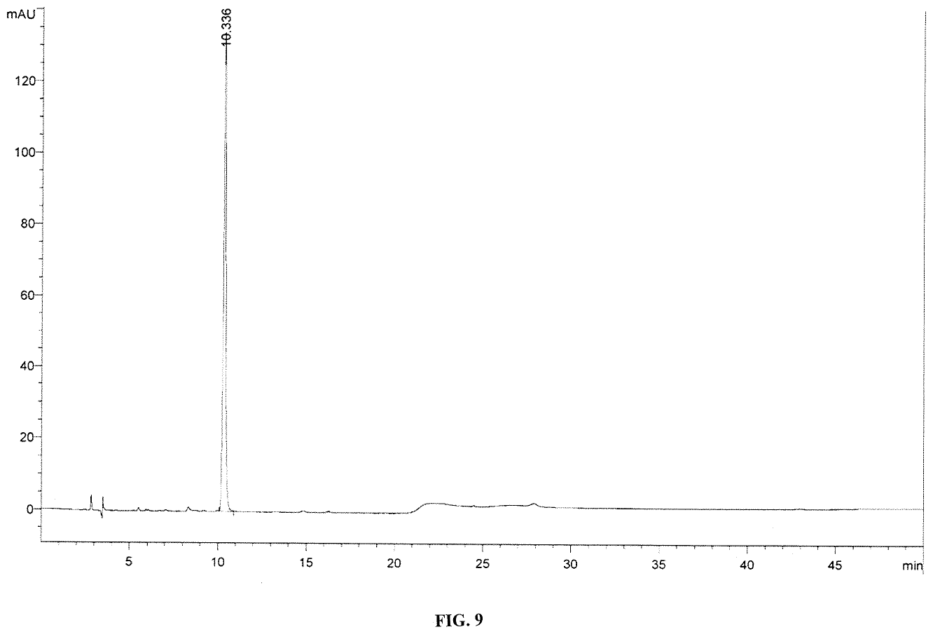

The reaction mixture of EXAMPLE 5, after 24 hrs, was inactivated by adjusting the pH to pH 5.5 with H₃PO₄ and then boiled for 10 minutes. After boiling the reaction mixture was filtered and diluted with RO water to 5% solids content. The diluted solution was passed through 1 L column packed with YWD03 macroporous adsorption resin (Cangzhou Yuanwei, China). Adsorbed steviol glycosides were eluted with 5 L 70% ethanol. The obtained eluate was evaporated until dryness to yield 16 g of dry powder which was dissolved in 80 mL of 70% methanol. The solution was crystallized at 20° C. for 3 days. The crystals were separated by filtration and dried in vacuum oven at 80° C. for 18 hours to yield 10.4 g of pure reb AM crystals with 95.92% purity, determined by HPLC assay. The chromatogram of reb AM is shown in FIG. 9. Those with skill in the art will appreciate that retention times can occasionally vary with changes in solvent and/or equipment.

### Example 8

Structure Elucidation of Rebaudioside AM

NMR experiments were performed on a Bruker 500 MHz spectrometer, with the sample dissolved in pyridine-d5. Along with signals from the sample, signals from pyridine-d5 at δ_{C} 123.5, 135.5, 149.9 ppm and δ_{H} 7.19, 7.55, 8.71 ppm were observed.

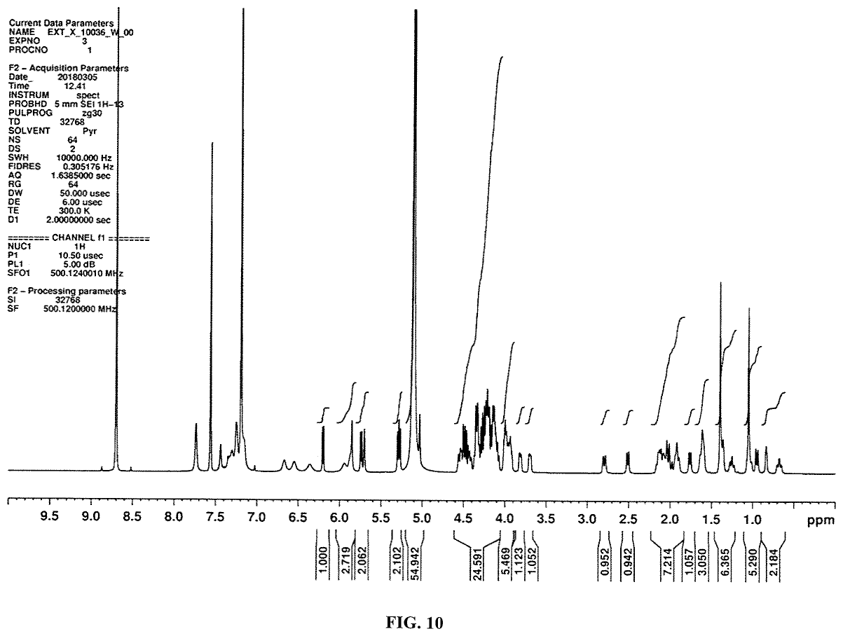

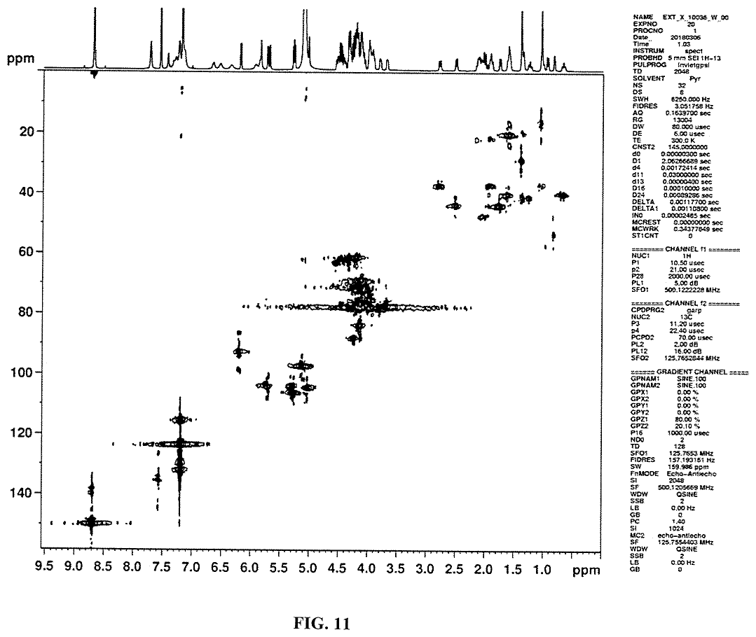

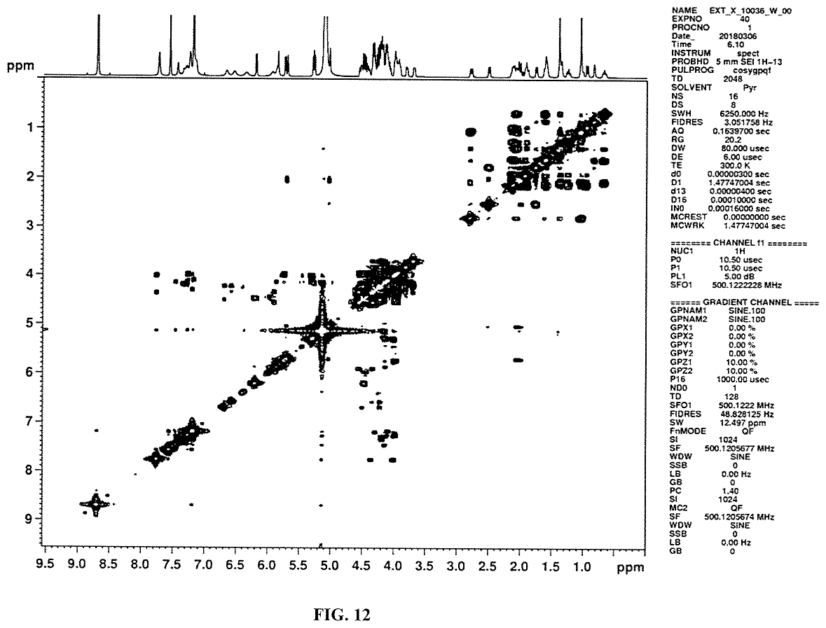

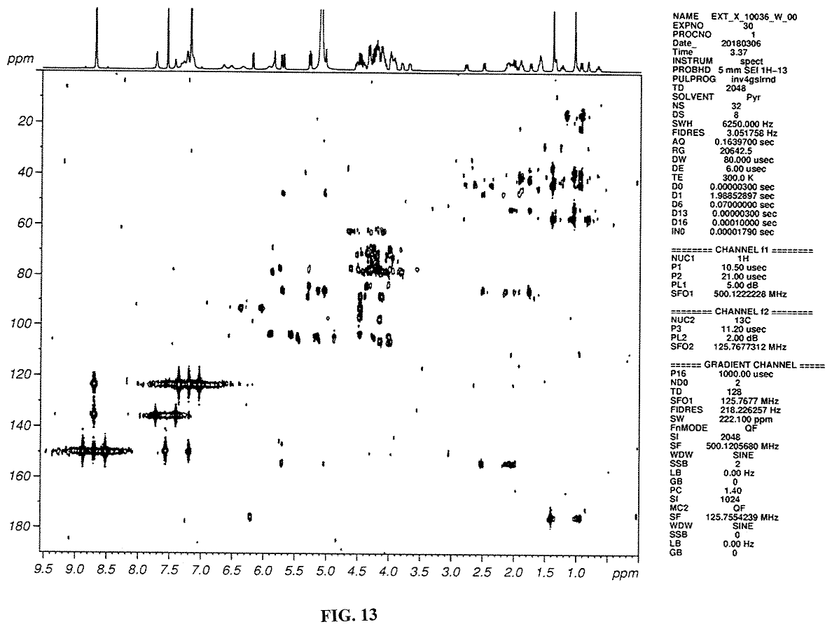

¹H-NMR-spectrum of rebaudioside AM in pyridine-d₅ reveal the excellent quality of the sample (see FIG. 10). The HSQC (see FIG. 11) shows the presence of an exo-methylene group in the sugar region with a long-range coupling to C-15, observable in the H,H—COSY (FIG. 12). Other deep-fielded signals of the quaternary carbons (C-13, C-16 and C-19) are detected by the HMBC (FIG. 13). Correlation of the signals in the HSQC, HMBC and H,H—COSY reveal the presence of steviol glycoside with the following aglycone structure:

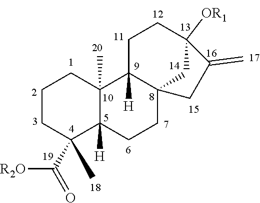

Correlation of HSQC and HMBC signals reveal five anomeric signals. The coupling constant of the anomeric protons of about 8 Hz and the broad signals of their sugar linkage allows the identification of these five sugars as β-D-glucopyranosides.

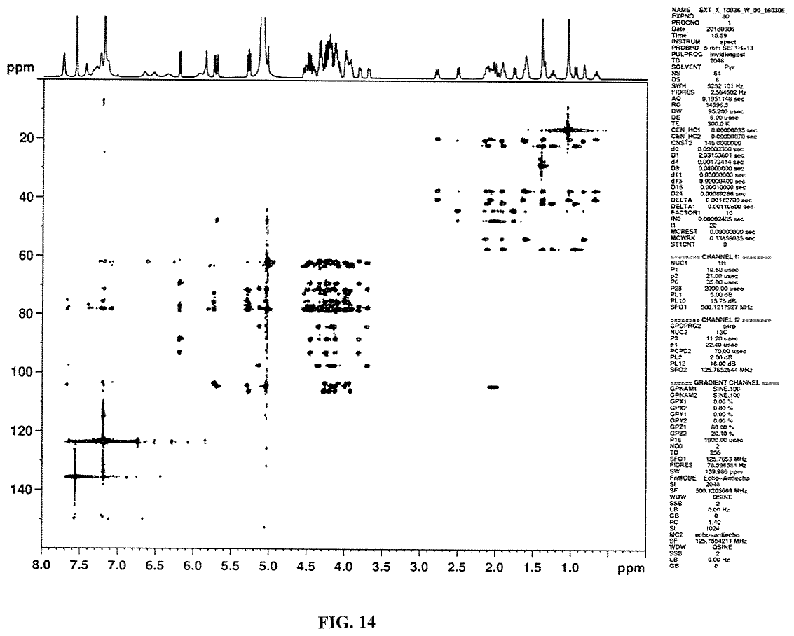

The observation of the anomeric protons in combination with HSQC and HMBC reveal the sugar linkage and the correlation to the aglycone. The assignment of the sugar sequence was confirmed by using the combination of HSQC-TOCSY (FIG. 14) and HSQC.

The NMR experiments above were applied to assign the chemical shifts of the protons and carbons, main coupling constants and main HMBC correlations (see Table 5).

TABLE 5
Chemical shifts of rebaudioside AM

| | | | | |
|---|---|---|---|---|
| Position | δ_{C} [ppm] | δ_{H} [ppm] | J [Hz] | HMBC (H → C) |

Aglycone moiety

| 1 | 39.9 | t | 0.68 | m | | |
|---|---|---|---|---|---|---|
| | | | 1.64 | m | | |
| 2 | 19.4 | t | 1.39 | m | | |
| | | | 2.08 | m | | |
| 3 | 37.4 | t | 1.05 | m | | |
| | | | 2.80 | m | | |
| 4 | 44.2 | s | — | | | |
| 5 | 57.3 | d | 0.95 | m | | |
| 6 | 21.7 | t | 1.90 | m | | |
| | | | 2.12 | m | | |
| 7 | 41.0 | t | 1.26 | m | | |
| | | | 1.38 | m | | |
| 8 | 41.9 | s | — | | | |
| 9 | 53.3 | d | 0.85 | m | | |
| 10 | 39.2 | s | — | | | |
| 11 | 20.1 | t | 1.59 | m | | |
| | | | 1.61 | m | | |
| 12 | 36.9 | t | 1.65 | m | | |
| | | | 1.92 | m | | |
| 13 | 85.9 | s | — | | | |
| 14 | 43.8 | t | 1.78 | d | 11.0 | |
| | | | 2.52 | d | 11.0 | |
| 15 | 47.4 | t | 2.00 | d | 16.0 | 7, 8, 9, 14 |
| | | | 2.06 | d | 16.0 | |
| 16 | 154.6 | s | — | | | |
| 17 | 104.3 | t | 5.03 | br s | | 13, 15, 16 |
| | | | 5.71 | br s | | |
| 18 | 28.5 | q | 1.40 | s | | 3, 4, 5, 19 |
| 19 | 175.2 | s | — | | | |
| 20 | 16.2 | q | 1.06 | s | | 1, 5, 9, 10 |

Sugar moiety
Sugar I: β-D-Glucopyranoside

| | | | | | | |
|---|---|---|---|---|---|---|
| 1ⁱ | 97.5 | d | 5.13 | d | 7.7 | 13 |
| 2ⁱ | 84.0 | d | 4.14 | m | | |
| 3ⁱ | 77.6 | d | 4.20 | m | | |
| 4ⁱ | 71.3 | d | 4.19 | m | | |
| 5ⁱ | 77.6 | d | 3.70 | m | | |
| 6ⁱ | 62.0 | t | 4.23 | m | | |
| | | | 4.32 | m | | |

Sugar II: β-D-Glucopyranoside

| | | | | | | |
|---|---|---|---|---|---|---|
| 1ⁱⁱ | 106.3 | d | 5.26 | d | 8.0 | 2* |
| 2ⁱⁱ | 76.8 | d | 4.13 | m | | |
| 3ⁱⁱ | 77.3 | d | 4.21 | m | | |
| 4ⁱⁱ | 71.6 | d | 4.18 | m | | |
| 5ⁱⁱ | 77.9 | d | 3.91 | m | | |
| 6ⁱⁱ | 62.4 | t | 4.29 | m | | |
| | | | 4.41 | m | | |

Sugar III: β-D-Glucopyranoside

| | | | | | | |
|---|---|---|---|---|---|---|
| 1ⁱⁱⁱ | 92.9 | d | 6.20 | d | 8.1 | 19 |
| 2ⁱⁱⁱ | 77.0 | d | 4.46 | m | | |
| 3ⁱⁱⁱ | 88.1 | d | 4.24 | m | | |
| 4ⁱⁱⁱ | 69.0 | d | 4.12 | m | | |
| 5ⁱⁱⁱ | 78.4 | d | 3.82 | m | | |
| 6ⁱⁱⁱ | 61.3 | t | 4.20 | m | | |
| | | | 4.33 | m | | |

Sugar IV: β-D-Glucopyranoside

| | | | | | | |
|---|---|---|---|---|---|---|
| 1^{iv} | 103.4 | d | 5.73 | d | 7.7 | 2ⁱⁱⁱ |
| 2^{iv} | 75.4 | d | 3.98 | m | | |
| 3^{iv} | 78.1 | d | 4.09 | m | | |
| 4^{iv} | 72.6 | d | 4.08 | m | | |
| 5^{iv} | 77.4 | d | 3.92 | m | | |
| 6^{iv} | 62.9 | t | 4.32 | m | | |
| | | | 4.51 | m | | |

Sugar V: β-D-Glucopyranoside

| | | | | | | |
|---|---|---|---|---|---|---|
| 1^{v} | 104.4 | d | 5.29 | d | 8.1 | 3ⁱⁱⁱ |
| 2^{v} | 75.1 | d | 4.00 | m | | |
| 3^{v} | 78.2 | d | 4.24 | m | | |
| 4^{v} | 71.4 | d | 4.27 | m | | |
| 5^{v} | 78.2 | d | 3.99 | m | | |
| 6^{v} | 61.9 | t | 4.27 | m | | |
| | | | 4.48 | m | | |

Correlation of all NMR data indicates rebaudioside AM having five β-D-glucopyranoses attached to a steviol aglycone, as depicted with the following chemical structure:

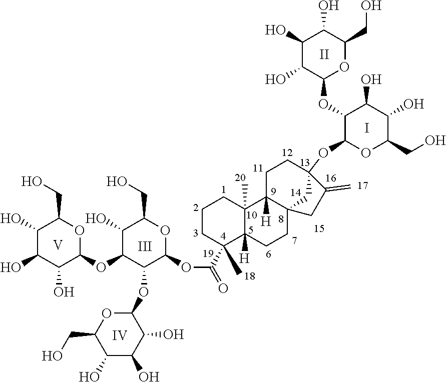

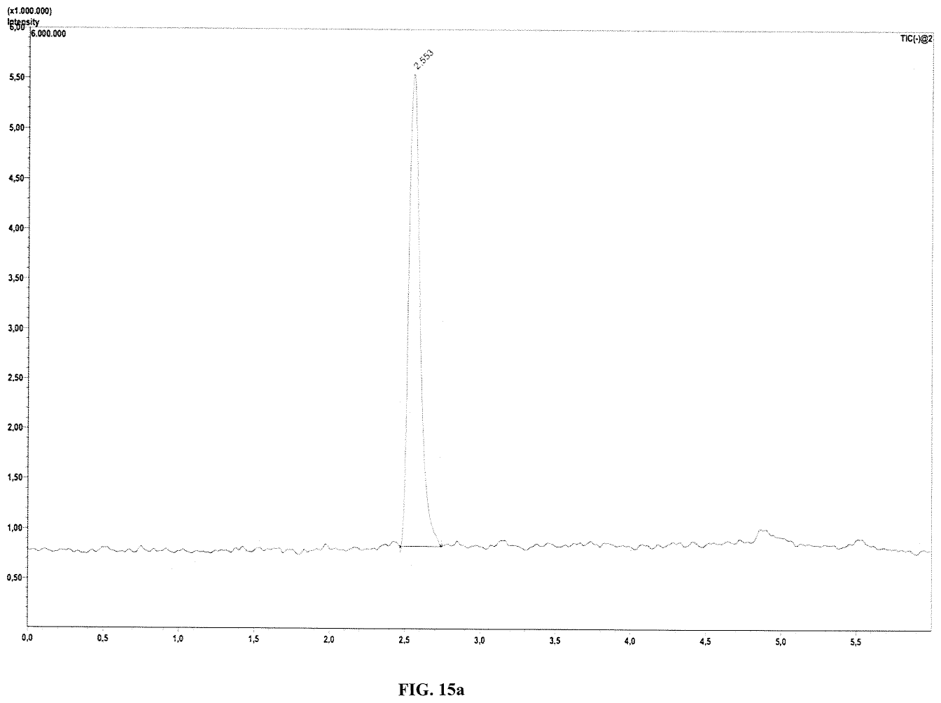

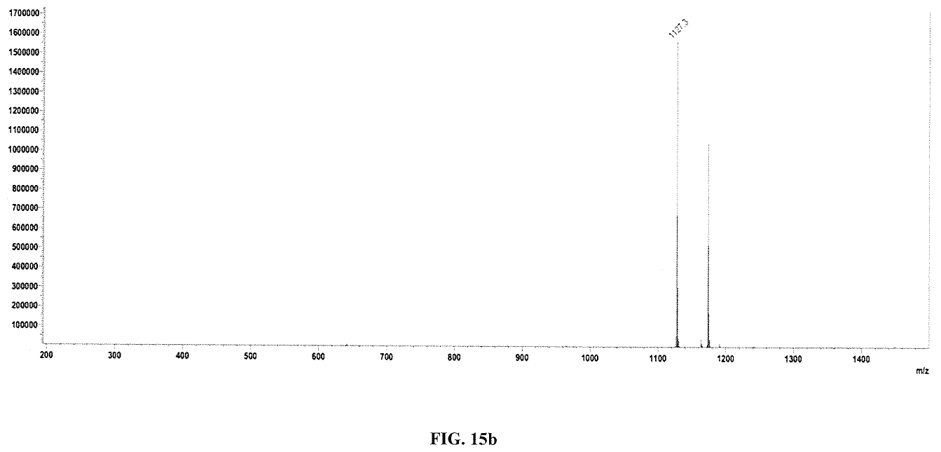

The chemical formula of rebaudioside AM is C₅₀H₈₀O₂₈, which corresponds to a calculated monoisotopic molecular mass of 1128.5. For LCMS analysis, rebaudioside AM was dissolved in methanol and analyzed using Shimadzu Nexera 2020 UFLC LCMS instrument on a Cortecs UPLC C18 1.6 μm, 50×2.1 mm column. The observed LCMS (negative ESI mode) result of 1127.3 (see FIG. 15a and FIG. 15b respectively) is consistent with rebaudioside AM and corresponds to the ion (M-H)⁻.

We claim:

1. Rebaudioside AM with the formula:

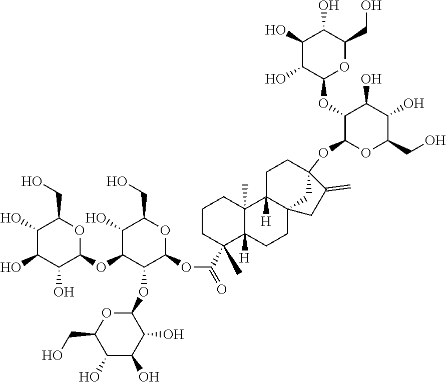

2. A method for producing a highly purified rebaudioside AM of claim 1, comprising the steps of:
a. providing a starting composition comprising an organic compound with at least one carbon atom;
b. providing a biocatalyst selected from the group consisting of an enzyme preparation, a cell or a microorganism; said biocatalyst comprising at least one enzyme capable of converting the starting composition to rebaudioside AM;
c. contacting the biocatalyst with a medium containing the starting composition to produce a medium comprising rebaudioside AM.

3. The method of claim 2 further comprising the step of:
d. separating the rebaudioside AM from the medium to provide a highly purified rebaudioside AM composition.

4. The method of claim 2, wherein the starting composition is selected from the group consisting of steviol, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, other steviol glycosides, polyols, carbohydrates, and combinations thereof.

5. The method of claim 2, wherein the microorganism is selected from the group consisting of E. coli, Saccharomyces sp., Aspergillus sp., Pichia sp., Bacillus sp., and Yarrowia sp.

6. The method of claim 2, wherein the enzyme is selected from the group consisting of: a steviol biosynthesis enzyme, a UDP glucosyltransferase, a UDP glucose recycling enzyme, a mevalonate (MVA) pathway enzyme, a 2-C-methyl-D-erythritol-4-phosphate pathway (MEP/DOXP) enzyme, geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase, UGT74G1, UGT85C2, UGT91D2, EUGT11, UGTS12, UGT76G1, or mutant variant thereof having >85% amino-acid sequence identity, >86% amino-acid sequence identity, >87% amino-acid sequence identity, >88% amino-acid sequence identity, >89% amino-acid sequence identity, >90% amino-acid sequence identity, >91% amino-acid sequence identity, >92% amino-acid sequence identity, >93% amino-acid sequence identity, >94% amino-acid sequence identity, >95% amino-acid sequence identity, >96% amino-acid sequence identity, >97% amino-acid sequence identity, >98% amino-acid sequence identity, >99% amino-acid sequence identity; and combinations thereof.

7. The method of claim 3, wherein the rebaudioside AM content in highly purified rebaudioside AM composition is greater than about 95% by weight on a dry basis.

8. A consumable product comprising rebaudioside AM of claim 1, wherein the product is selected from the group consisting of a food, a beverage, a pharmaceutical composition, a tobacco product, a nutraceutical composition, an oral hygiene composition, and a cosmetic composition.

9. The consumable product of claim 8, further comprising at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, flavonoids, alcohols, polymers and combinations thereof.

10. The consumable product of claim 8, further comprising at least one functional ingredient selected from the group consisting of saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

11. The consumable product of claim 8, further comprising a compound selected from the group consisting of rebaudioside A, rebaudioside A2, rebaudioside A3, rebaudioside B, rebaudioside C, rebaudioside C2, rebaudioside D, rebaudioside D2, rebaudioside E, rebaudioside E2, rebaudioside E3, rebaudioside F, rebaudioside F2, rebaudioside F3, rebaudioside G, rebaudioside H, rebaudioside I, rebaudioside I2, rebaudioside I3, rebaudioside J, rebaudioside K, rebaudioside K2, rebaudioside KA, rebaudioside L, rebaudioside M, rebaudioside M2, rebaudioside N, rebaudioside O, rebaudioside O2, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside W, rebaudioside W2, rebaudioside W3, rebaudioside Y, rebaudioside Z1, rebaudioside Z2, dulcoside A, dulcoside C, rubusoside, steviolbioside, steviolbioside A, steviolbioside B, steviolmonoside, steviolmonoside A, stevioside, stevioside A, stevioside B, stevioside C, stevioside D, stevioside E, stevioside E2, stevioside F, NSF-02, Mogroside V, Luo Han Guo, allulose, D-allose, D-tagatose, erythritol, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside, sucralose, potassium acesulfame, aspartame, alitame, saccharin, cyclamate, neotame, dulcin, suosan advantame, gymnemic acid, hodulcin, ziziphin, lactisole, glutamate, aspartic acid, glycine, alanine, threonine, proline, serine, lysine, tryptophan, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols, sugar alcohols, L-sugars, L-sorbose, L-arabinose, trehalose, galactose, rhamnose, various cyclodextrins, cyclic oligosaccharides, various types of maltodextrins, dextran, sucrose, glucose, ribulose, fructose, threose, xylose, lyxose, altrose, mannose, idose, lactose, maltose, invert sugar, isotrehalose, neotrehalose, isomaltulose, erythrose, deoxyribose, gulose, talose, erythrulose, xylulose, cellobiose, amylopectin, glucosamine, mannosamine, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, isomerized liquid sugars such as high fructose corn syrups, coupling sugars, soybean oligosaccharides, D-psicose, D-ribose, L-glucose, L-fucose, D-turanose, D-leucrose.

12. A method for modifying the flavor of a consumable comprising the steps of:
a. providing a consumable; and
b. adding a flavor with modifying properties comprising rebaudioside AM of claim 1,
wherein rebaudioside AM is present in the consumable at a concentration at or below the detection threshold of the flavor with modifying properties.

13. The method of claim 12, wherein modifying the flavor comprises enhancing the sweetness of the consumable.

14. A method for suppressing foaming of a beverage or food product, comprising
a. providing a beverage or a food product, and
b. adding a foam suppressor comprising rebaudioside AM of claim 1.


SEQUENCE LISTING (3 sequences)

SEQ ID NO: 1 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLDFLRLHSHQGKNLMLSEKIQNLNTLQHTLRKAEEYLAELKSETLYEEFEAKFEEIGLERGWGDNAERVLDMIRLLLDLLEAPDPSTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENDEHLCVLKDKKKPILFTMARLDRVKNLSGLVEWYGKNTRLRELVNLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADLLADFFTKCKEDPSHWDEISKGGLQRIEEKYTWQIYSQRLLTLTGVYGFWKHVSNLDRLEHRRYLEMFYALKYRPLAQAVPLAQDD

SEQ ID NO: 2 - protein, 442 aa, Solanum lycopersicum
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQGIPAGKLLVSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKPENSTVFVSFGSEYFLSKEDMEEIAFALEASNVNFIWVVRFPKGEERNLEDALPEGFLERIGERGRVLDKFAPQPRILNHPSTGGFISHCGWNSVMESIDFGVPIIAMPIHNDQPINAKLMVELGVAVEIVRDDDGKIHRGEIAEALKSVVTGETGEILRAKVREISKNLKSIRDEEMDAVAEELIQLCRNSNKSK

SEQ ID NO: 3 - protein, 458 aa, Stevia rebaudiana
MENKTETTVRRRRRIILFPVPFQGHINPILQLANVLYSKGFAITILHTNFNKPKTSNYPHFTFRFILDNDPQDERISNLPTHGPLAGMRIPIINEHGADELRRELELLMLASEEDEEVSCLITDALWYFAQDVADSLNLRRLVLMTSSLFNFHAHVSLPQFDELGYLDPDDKTRLEEQASGFPMLKVKDIKSAYSNWQIGKEILGKMIKQTKASSGVIWNSFKELEESELETVIREIPAPSFLIPLPKHLTASSSSLLDHDRTVFEWLDQQAPSSVLYVSFGSTSEVDEKDFLEIARGLVDSGQSFLWVVRPGFVKGSTWVEPLPDGFLGERGKIVKWVPQQEVLAHPAIGAFWTHSGWNSTLESVCEGVPMIFSSFGGDQPLNARYMSDVLRVGVYLENGWERGEVVNAIRRVMVDEEGEYIRQNARVLKQKADVSLMKGGSSYESLESLVSYISSL